(19) United States
(12) Patent Application Publication
(10) Pub. No.: US 2023/0181783 A1
(43) Pub. Date: Jun. 15, 2023

(54) PLASMA DEVICE

(71) Applicant: UNIVERSITY OF LANCASTER, Lancaster, Lancashire (GB)

(72) Inventors: Bhagirath GHIMIRE, Lancaster (GB); Robert David SHORT, Carnforth (GB)

(21) Appl. No.: 17/924,746

(22) PCT Filed: May 13, 2021
(86) PCT No.: PCT/GB2021/051154

§ 371 Date: Nov. 11, 2022

(30) Foreign Application Priority Data
May 13, 2020 (GB) 2007023.1

(51) Int. Cl. A61L 2/26; H05H 1/24; A61L 2/00; A61L 2/14

(52) U.S. Cl. CPC A61L 2/26; H05H 1/245; A61L 2/0011; A61L 2/14; H05H 2245/34

(57) ABSTRACT

There is described herein, a plasma device for the generation of oxygen and nitrogen species (RONS) and methods of generating RONS using the plasma device.

The present invention relates to a plasma device and method for the generation of hydrogen peroxide. There is also provided a plasma treatment method for a patient in need thereof including activating a hydrogel through contact with a plasma including hydrogen peroxide to release therapeutic agents from the hydrogel dressing. There is also provided a method of deactivation of micro-organisms (bacteria and viruses) on surfaces/materials that are thermally sensitive. In such embodiments, the plasma including hydrogen peroxide may be used to decontaminate or treat surfaces directly.

### BACKGROUND TO THE INVENTION

US20170312376A1 discloses a hydrogen peroxide plasma ionisation generator device. However, this document does not disclose the production of hydrogen peroxide by using plasma. The role of plasma in this invention is to ionize hydrogen peroxide (in vapour form) and air into finer sized particles, although there is no detail about the associated mechanism.

US20170312376A1 makes use of the hydrogen peroxide obtained through commercial manufacturing. This is then mixed with air, and further ionized by plasma to produce reactive oxygen species (ROS).

The use of a commercial hydrogen peroxide generator is associated with a number of drawbacks, (i) the mixture of hydrogen and oxygen used in this process is highly combustible and high safety precautions are always necessary; (ii) the generated hydrogen peroxide comes with impurities (and also added promoters/stabilizers) which pose risks in biomedical applications; (iii) the transport, storage and handling of bulk hydrogen peroxide involves hazards and escalating expenses suggesting the urgent need of on-site/on demand generation.

US20170312376A1 refers to a device including two nozzles where either liquid hydrogen peroxide (N1) or liquid hydrogen peroxide+air (N2) exit from either nozzle. A “plasma generator device” is only installed at the only end of nozzle N2.

US2017/0050039 discloses a plasma treatment method comprising providing a hydrogel screen between a plasma source and the surface to be treated. This hydrogel limits/blocks highly reactive/dangerous short-lived reactive species including hydroxyl radicals from reaching the target and allows the transmission of long-lived reactive species including hydrogen peroxide onto the target with beneficial therapeutic effects.

The production of reactive species including hydrogen peroxide may be achieved by using commercially available plasma devices (such as those sold under the trade marks kINPen®, Adtec® Sterilas® and PlasmaDerm®). All have drawbacks compared to the invention disclosed herein. In particular, the device and method of the present invention allow the production of reactive oxygen nitrogen species (RONs) including hydrogen peroxide with minimal or no heating or dehydration of a sample which is contacted by the resultant RONs composition.

The kINPenMED cannot operate at lower gap distances due to its required high argon flow rate (in the range of around 3-5 litres per minute). Further it poses challenges in the treatment of delicate materials (in part due to the high gas flow rate damaging the target). The high rate of argon used represents another (economic) drawback. Also, as a result of the high argon flow rate, the evaporation of the liquid from any target occurs at a high rate, and possibly before sufficient (therapeutically-useful) quantities of hydrogen peroxide are formed.

Though the Adtec® Sterilas® plasma torch could generate hydrogen peroxide over a wider area than the kINPenMED® device, the concentration may not be sufficient (and is certainly less than the claimed device). The Sterilas® device has a high operating temperature meaning that an additional cooling unit is required before the resultant composition can be applied to treat thermally sensitive materials.

The PlasmaDerm® device is operated in ambient air gas. It produces relatively high quantities of ozone and nitrogen—derivatives, such as nitrogen oxides (NOxs), Such derivatives tend to be produced in greater abundance than hydrogen peroxide.

In summary, none of these devices are specifically optimized for the production of RONs such as hydrogen peroxide and have restrictions in their applications on thermally-sensitive material, including human tissue such as wounds.

The device and method of the present invention allows the high concentration delivery of hydrogen peroxide at relatively low temperatures over a wide area. The device and method of the present invention allow production and delivery of sufficient/high concentration of RONS such as hydrogen peroxide and nitrous acid over large surface areas whilst achieving control over the delivered plasma-gas temperature. This allows the decontamination or treatment of thermally sensitive materials, whilst avoiding damage or dehydration of the thermally sensitive materials. The temperature of the plasma jet comprising RONS emitted from the device of the present invention is generally maintained at less than 40° C.

The methods and apparatus of the present invention allow the production of a plasma including hydrogen peroxide over a wide surface area, allowing the delivery of sufficient hydrogen peroxide to activate a hydrogel coating and release therapeutic agents. The temperature of the plasma is controlled to allow it to be cool enough to safely contact thermally sensitive materials, including human tissue whilst avoiding or minimising the risk of overheating or dehydration of the thermally sensitive material. The plasma generated can be in sustained contact with tissue/thermally sensitive materials for relatively long periods (˜1-2 mins) without the need to raster over the surface.

The production and delivery of compositions including high concentrations of RONS and relatively low temperatures is achievable through the use of the apparatus described herein.

The concentration and temperature of the compositions generated may be influenced by various factors including:
the ratio of the inner diameter or inner width of the or each conduit (Di) to the spacing between the first and second electrodes (d);
the inclusion of more than one electrode connected to the negative terminal of the power supply or the ground;
the ratio of the inner diameter or inner width of the or each conduit (Di) to the spacing between the electrode connected to the positive terminal of the power supply and the ground electrode furthest from it (d2);
the parameters used to operate the plasma such as applied voltage, frequency, gas flow, etc.

### SUMMARY OF INVENTION

According to a first aspect of the present invention, there is provided a plasma device for the generation of reactive oxygen nitrogen species (RONS) comprising:
a reservoir containing a carrier gas;
a housing in fluid communication with the reservoir wherein the reservoir includes air or water, or the housing includes an air or water inlet;
first and second electrodes spaced along the housing wherein a plasma generation zone is provided in the spacing between the first and second electrodes;
a power supply suitable to apply an electrical potential between the first and second electrodes sufficient to form a plasma through the ionisation of the carrier gas, and to form hydrogen peroxide from oxygen and hydrogen in the air or in the water;
at least one outlet for the plasma and the hydrogen peroxide formed.

The device of the present invention allows generation of hydrogen peroxide with a non-thermal plasma through the ionisation of ambient water molecules. The device of the present invention allows the generation of plasma over a wide surface area and allows the delivery of sufficient hydrogen peroxide to release therapeutic agents from a hydrogel. The temperature of the plasma is controlled to avoid heating and dehydration of the hydrogel.

According to a further aspect of the present invention, there is provided a plasma device for the generation of reactive oxygen nitrogen species (RONS) comprising:
a reservoir containing a carrier gas;
a housing in fluid communication with the reservoir wherein the reservoir includes air or water, or the housing includes an air or water inlet, said housing comprising one or more conduits formed from a dielectric material;
first and second electrodes spaced along the at least one conduit wherein a plasma generation zone is provided in the spacing between the first and second electrodes;
a power supply suitable to apply an electrical potential between the first and second electrodes sufficient to form a plasma through the ionisation of the carrier gas, and to form hydrogen peroxide from the water molecules;
at least one outlet for the plasma and the hydrogen peroxide formed.

According to one embodiment, one of the first and second electrodes is a ground electrode, and the device includes more than one ground electrode, suitably two or three ground electrodes. Suitably, the inner and outer surfaces of the one or more conduits are formed from the dielectric material.

According to one embodiment, the first electrode is connected to the positive terminal of the power supply and the second electrode is connected to the negative terminal of the power supply, and wherein the first electrode is provided within the conduit, and the second electrode is provided externally to the conduit, generally abutting an outer wall of the conduit. Where the device includes more than one electrode connected to the negative terminal of the power supply, each may be provided externally to the conduit, generally abutting an outer wall of the conduit.

Generally the device includes a flowmeter between the reservoir and the housing, configured to control the flow of carrier gas from the reservoir to the housing.

According to a further aspect of the present invention, there is provided a method of forming a plasma including reactive oxygen nitrogen species (RONS) comprising:
providing a device as described herein;
providing a flow of the carrier gas and air or water through the housing;
applying an electrical potential between the first and second electrodes to ionise the carrier gas to form a plasma and to ionise water and/or air to form reactive oxygen nitrogen species.

Generally the method involves the ionisation of water molecules to form hydrogen peroxide.

Generally the ambient water molecules are included in the carrier gas.

Where air is present in the plasma generation zone, air and water may be ionised to form one or more of ozone, nitrous acid, peroxynitrate, hydrogen peroxide, etc.

According to a further aspect of the present invention, there is provided a method of forming a plasma including RONS comprising:
providing a device as described herein;
providing a flow of the carrier gas through the housing wherein the carrier gas includes water molecules;
applying an electrical potential between the first and second electrodes to ionise the carrier gas to form a plasma and RONS to ionise the water molecules to form hydrogen peroxide.

According to a further embodiment, there is provided a plasma treatment method for a patient in need thereof comprising:
providing a hydrogel on an anatomical region of interest of the patient;
generating a plasma comprising RONS using the device described herein, and/or using the method described herein;
contacting a surface of the hydrogel with the plasma comprising RONS;
wherein contact of the hydrogel with the plasma activates the hydrogel dressing.

Suitably the hydrogel dressing has a thickness of 1 to 50 mm. Generally, the hydrogel includes therapeutic agents and contact of the hydrogel with the plasma activates release of the therapeutic agents (generally antimicrobial agents) from the hydrogel.

According to a further embodiment, there is provided a system including the device as described herein and a hydrogel dressing comprising therapeutic agents wherein the hydrogel dressing is activatable upon contact with a plasma comprising RONS such as hydrogen peroxide to release the therapeutic agents.

According to a further embodiment there is provided a kit of parts comprising the plasma device described herein and instructions for use. Generally the kit includes one or more hydrogel compositions as described herein.

Throughout the Application, where a device is described as having, including, or comprising specific components, or where a method is described as having, including, or comprising specific process steps, it is contemplated that the device of the present teachings also consist essentially of, or consist of, the recited components, and that the method of the present teachings also consist essentially of, or consist of, the recited process steps.

In the Application, where an element or component is said to be included in and/or selected from a list of recited elements or components, or where a method is said to include certain steps, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

It should be understood that the expression “at least one of” includes individually each of the recited objects after the expression and the various combinations of two or more of the recited objects unless otherwise understood from the context and use.

The use of the singular herein, for example, “a,” “an,” and “the,” includes the plural (and vice versa) unless specifically stated otherwise.

The use of the terms “include,” “includes”, “including,”, “comprise”, “comprises” “comprising”, “have,” “has,” or “having” should be generally understood as open-ended and non-limiting unless specifically stated otherwise.

The use of the singular herein includes the plural (and vice versa) unless specifically stated otherwise. All numerical values provided incorporate 10% less than and 10% more than the numerical value provided.

In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

Features, integers, characteristics, or groups described in conjunction with a particular aspect, embodiment or example of the invention are to be understood to be applicable to any other aspect, embodiment or example described herein unless incompatible therewith.

At various places in the present specification, values are disclosed in groups or in ranges. It is specifically intended that the description include each and every individual sub combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as,” “including,” or “for example,” is intended merely to illustrate better the present teachings and does not pose a limitation on the scope of the invention unless claimed. No language in the specification should be construed as indicating any non-claimed element as essential to the practice of the present teachings.

A person skilled in art could make a suitable choice of the dielectric material for use in the present device by assessing the characteristics of the material. The dielectric constant (k) of the materials used in the current device are generally in the range of from 3-10. Commonly used dielectric materials are fused quartz (k=3.8), glass (k=6), alumina (k=9), epoxy (k=4.8), and polypropylene (k=3.9), etc.

Device

According to a first aspect of the present invention, there is provided a plasma device for the generation of RONS such as hydrogen peroxide comprising:
a reservoir containing a carrier gas;
a housing in fluid communication with the reservoir wherein the reservoir includes air or water, or the housing includes an air or water inlet;
first and second electrodes spaced along the housing wherein a plasma generation zone is provided in the spacing between the first and second electrodes;
a power supply suitable to apply an electrical potential between the first and second electrodes sufficient to form a plasma through the ionisation of the carrier gas, and to form RONS such as hydrogen peroxide from oxygen and hydrogen in the air or in the water;
at least one outlet for the plasma and the hydrogen peroxide formed.

According to a further aspect of the present invention, there is provided a plasma device for the generation of RONS such as hydrogen peroxide comprising:
a reservoir containing a carrier gas;
a housing in fluid communication with the reservoir, wherein the reservoir includes air or water, or the housing includes an air or water inlet, said housing comprising one or more conduits formed from a dielectric material;
first and second electrodes spaced along the at least one conduit wherein a plasma generation zone is provided in the spacing between the first and second electrodes;
a power supply suitable to apply an electrical potential between the first and second electrodes sufficient to form a plasma through the ionisation of the carrier gas, and to form RONS such as hydrogen peroxide from the water molecules;
at least one outlet for the plasma and the hydrogen peroxide formed.

Generally, the electrode connected to the negative terminal of the power supply (ground electrode) is fully isolated from the interstice defined by the walls of the conduit. Suitably the electrode connected to the negative terminal of the power supply (ground electrode) is provided externally to the conduit, generally abutting an outer wall of the conduit, typically provided around the outer walls of the conduit.

Typically the inner and outer surfaces of the conduit(s) are formed from the dielectric material.

The present invention makes use of plasma to ionize ambient water molecules directly resulting in the formation RONS such as hydrogen peroxide. The concentration of the RONS such as hydrogen peroxide generated in situ/real time can be controlled by controlling the parameters of the device, for instance including changing the inter electrode spacing along the length of the conduit (longitudinal spacing), incorporation of additional ground electrodes, the ratio of the inner diameter or inner width of the or each conduit or the housing (Di) to the longitudinal spacing between the first and second electrodes, and altering the dimensions of the housing. The device of the present invention may include a plurality of conduits, each including a plasma generation zone, resulting in a jet array for the production of RONS such as hydrogen peroxide. The conduits may be arranged in linear, coiled, or helical configurations or some or all of the conduits may undulate in a wave form along an axis of the plasma device extending in a longitudinal direction, for instance in a sine shaped wave form.

According to one embodiment, one of the first and second electrodes is a ground electrode, and the device includes more than one ground electrode. The inclusion of more than one ground electrode enables the plasma device to be operated at lower temperatures than conventional plasma devices, in particular at around room temperature, whilst generating plasma with a higher concentration of hydrogen peroxide than conventional plasma devices. The incorporation of a second ground electrode, is associated with the reduction of ignition voltage and an increase in the length of plasma plume outside the tube. In addition, the large spacing along the length of the conduit provided between the electrode connected to the positive terminal of the power supply and the ground electrode furthest from it (spacing referred to as d2) provides a higher region for the stronger dissociation of water vapour molecules which tends to result in the production of a higher concentration of hydrogen peroxide.

Where more than one ground electrode is provided, each of the ground electrodes is generally fully isolated from the interstice defined by the walls of the conduit. Suitably each of the ground electrodes is provided externally to the conduit, for instance around an outer surface/wall of the conduit. Typically, each of the ground electrodes is spaced along an outer surface of the conduit.

The electrode connected to the positive terminal of the power supply is generally provided within the conduit. The positive electrode may be provided in the interstice between walls of the conduit and generally does not abut the inner surfaces of the conduit.

Where more than one ground electrode is included, inter-electrode spacing is used to refer to the distance along the length of the conduit between the electrode connected to the positive terminal of the power supply and the ground electrode closest to it is referred to as d1.

The first and second electrodes are generally provided within the housing. As noted above, the ground electrode is generally provided externally to the conduit, typically being provided around the outer walls of the conduit, and the electrode connected to the positive terminal of the power supply (high voltage electrode) is generally provided within the conduit. Where the device includes more than one ground electrode, the ground electrodes are generally spaced along the outer walls or surfaces of the conduit or conduit. The electrode connected to the positive terminal of the power supply is preferably provided within the housing. Generally all electrodes are provided within the housing. The provision of any of the electrodes externally to the housing could result in safety issues and should be avoided.

The plasma device of the present invention does not generally include any inlet for any reactive oxygen nitrogen species (RONS), including any inlet for hydrogen peroxide. Carrier gas, air and/or water molecules are fed into the housing, and RONS such as hydrogen peroxide are generated therein. Generally, the water molecules are present in the carrier gas. The air may be ambient air in the housing, or may be introduced through an inlet. Typically, the housing includes at most three inlets, firstly an inlet allowing fluid communication between the housing and the reservoir containing a carrier gas, optionally secondly the air or water inlet, and optionally an inlet between the second electrode and the outlet. Generally, the only species fed into the housing are the carrier gas, water and/or air; typically the only species fed into the housing is the carrier gas including water molecules. According to one embodiment, the reservoir containing a carrier gas includes water vapour molecules and the housing includes only one inlet.

Typically, the carrier gas, air and/or water are ionised simultaneously in the plasma generation zone to form plasma and reactive oxygen nitrogen species (RONS). Generally the carrier gas includes water vapour molecules and the carrier gas is ionised in the plasma generation zone to form plasma and hydrogen peroxide. Optionally, air is also ionised to form other RONS. Generally the plasma includes hydrogen peroxide.

Generally there is not an overlap of the first and second electrodes, for instance along the longitudinal axis of the housing or conduit. The plasma generation zone is provided in the longitudinal spacing between the first and second electrodes, and the generation zone is not in any area of overlap of the first and second electrodes.

In prior art plasma devices with a configuration including an overlap between the first and second electrodes, the electrodes either overlap, or are separated by a relatively small gap compared to the claimed device. Although this will ease the generation of plasma, it will also result in significant heating of the electrode(s) and/or housing as the electron density and gas temperature would be significantly higher in the overlapping region. This will increase with increasing length of the overlap region. This is addressed in the device of the present invention by arranging the electrodes in a non-overlapping configuration. With a configuration including no overlap between the first and second electrodes, the gas temperature of the system is generally lower.

As noted above, the housing typically includes one or more conduits formed from a dielectric material. The electrode connected to the positive terminal of the power supply (HV electrode) is generally provided within the or each conduit, the electrode connected to the negative terminal of the power supply (ground electrode) is generally provided externally to the conduit, and typically abuts an outer surface of the conduit. All electrodes are typically provided within the housing.

The housing may be in the form of a dielectric tube, or each conduit may be in the form of a dielectric tube. The device may include more than one dielectric tube, where the first and second electrodes may be spaced along each dielectric tube. The dielectric material is generally quartz, glass, alumina, PTFE, etc. Where present, the dielectric tube is in fluid communication with the reservoir, and the reservoir includes air or water. Alternatively or additionally, the carrier gas may include water molecules. Alternatively, the dielectric tube may include an air or water inlet. In such embodiments, the air or water inlet may be provided between the reservoir and the first electrode to allow mixing of the carrier gas and the air or water prior to delivery to the plasma generation zone. The dielectric tube(s) may be in communication with a flowmeter configured to control the flow of carrier gas from the reservoir to the housing.

The parameters of the device and method disclosed herein may be suitably adjusted to control the concentration of RONS such as hydrogen peroxide formed. Adjustable parameters include the longitudinal spacing between the first and second electrodes, the inner diameter or inner width of the conduit(s), the ratio between the inner diameter or inner width of the conduit(s) and the longitudinal spacing between the first and second electrodes, the length of the housing, the electric potential applied between the first and second electrodes, the flow rate of carrier gas, the flow rate of water molecules, and the flow rate of air.

Preferably the longitudinal spacing between the first and second electrodes is larger than that of standard plasma devices, for instance, where the device includes only one ground electrode, the inter-electrode longitudinal spacing (d) is generally greater than 20 mm, for instance greater than 2 cm to 100 cm. The inventors have surprisingly found that increasing the gap distance along the length of the conduit between the first electrode and the second electrode increases the formation of energetic streamers which effectively dissociate water molecules to form hydroxyl radicals, which subsequently combine to form RONS, in particular, hydrogen peroxide. Devices having an inter-electrode spacing of less than 20 mm produce far lower concentrations of RONS, in particular, hydrogen peroxide.

Where the device includes only one ground electrode, the inter-electrode spacing along the length of the conduit (d) is generally greater than 20 mm, for instance greater than 2 cm to 100 cm, and there is a longitudinal spacing between the one of the first and second electrodes closest to the outlet and the outlet of 0.5 cm or more. Typically, the inter-electrode spacing is 15 to 20 cm.

Where the device includes more than one ground electrode, the spacing between the electrode connected to the positive terminal of the power supply and the ground electrode closest thereto along the length of the conduit (d1) is generally 2 to 10 cm.

Where the device includes more than one ground electrode, the spacing between the electrode connected to the positive terminal of the power supply and the ground electrode furthest from it along the length of the conduit (d2) is generally 4 to 100 cm and there is a spacing between the one of the first and second electrodes closest to the outlet and the outlet of 0.5 cm or more.

According to one embodiment, the inter-electrode spacing along the length of the conduit is more than 10 cm, typically more than 50 cm, generally up to 100 cm. Discharges between electrodes at inter-electrode spacings of more than 5 cm is generally achieved in the device of the present invention through the inclusion of more than one ground electrode.

The housing or conduit may extend linearly along the central longitudinal axis of the device, extend helically around the central longitudinal axis, or may curve along the longitudinal axis, for instance undulating along the central longitudinal axis in a wave form, for instance a sine wave. Non-linear arrangements are of particular utility where the inter-electrode spacing is more than 50 cm.

Each conduit is defined by one or more walls formed from a dielectric material. Generally all of the surfaces of each conduit are formed from a dielectric material. Where the cross-section of the conduit is circular, the conduit has an associated inner diameter. Where the cross-section of the conduit is not circular, the conduit has an associated minimum inner width defined as the straight line segment that passes through the centre of the cross-section, and whose endpoints lie on the inner surface or surfaces. For simplicity, we refer hereinafter to an inner diameter or inner width.

According to one embodiment, the spacing along the length of the conduit between the first and second electrodes (d) is dependent on the inner diameter or inner width of the, or each conduit (Di). Generally Di is 0.05 to 1.5 cm, typically 0.05 to 1.2 cm.

Where the device includes a single ground electrode, the ratio of the inner diameter or inner width of the or each conduit (Di) to the spacing along the length of the conduit between the first and second electrodes (d) may be 0.0005 to 0.75, typically 0.001 to 0.6.

The ratio Di to d may be at least 0.001, generally 0.01 to 0.05, typically 0.02, suitably less than 0.05. Typically the ratio Di to d is less than 0.6, generally less than 0.5. The spacing along the length of the conduit between the first and second electrodes may be greater than 2 cm to 100 cm and there may be a spacing between the one of the first and second electrodes closest to the outlet and the outlet of 0.5 cm or more.

According to one embodiment, the device includes a single ground electrode, the spacing along the length of the conduit between the first and second electrodes (d) is 2 to 10 cm, the inner diameter of the, or each conduit (Di) is 0.05 to 1.2 cm, there is a spacing between the one of the first and second electrodes closest to the outlet and the outlet of 0.5 cm or more, and the ratio of Di/d is 0.005 to 0.6.

According to one embodiment, the device includes a single ground electrode, the spacing along the length of the conduit between the first and second electrodes (d) is 15 to 20 cm, the inner diameter of the, or each conduit (Di) is 0.05 to 1.2 cm, there is a spacing between the one of the first and second electrodes closest to the outlet and the outlet of 0.5 cm or more, and the ratio of Di/d is 0.0025 to 0.06.

According to one embodiment, the device includes a single ground electrode, the spacing along the length of the conduit between the first and second electrodes (d) is 50 to 100 cm, the inner diameter of the, or each conduit (Di) is 0.05 to 1.2 cm, there is a spacing between the one of the first and second electrodes closest to the outlet and the outlet of 0.5 cm or more, and the ratio of Di/d is 0.0005 to 0.024.

According to one embodiment, the spacing along the length of the conduit between the first and second electrodes is more than 2 to 100 cm, there is a spacing between the one of the first and second electrodes closest to the outlet and the outlet of at least 0.5 cm, the or each conduit has an associated inner diameter or minimum inner width, and the ratio of the inner diameter or inner width of the or each conduit (Di) to the spacing between the first and second electrodes (d) is 0.0005 to 0.6, suitably wherein the spacing between the first and second electrodes is 15 to 20 cm and the ratio Di/d is 0.002 to 0.08. Suitably, the inter-electrode spacing is more than 2 cm and up to 20 cm and the ratio Di to d is 0.0025 to 0.6. Alternatively, the inter-electrode spacing is 10 to 30 cm and the ratio Di to d is 0.001 to 0.12; the inter-electrode spacing is 30 to 50 cm and the ratio Di to d is 0.001 to 0.04; or the inter-electrode spacing is 50 to 100 cm and the ratio Di to d is 0.00025 to 0.03, generally 0.0005 to 0.024.

According to one embodiment, the device includes more than one ground electrode, the spacing along the length of the conduit between the electrode connected to the positive terminal of the power supply and the ground electrode closest thereto (d1) is around 2 cm, the inner diameter of the, or each conduit (Di) is 0.05 to 1.2 cm, and the ratio of Di/di is 0.025 to 0.6.

According to one embodiment, the device includes more than one ground electrode, the spacing along the length of the conduit between the electrode connected to the positive terminal of the power supply and the ground electrode closest thereto (d1) is 3 to 6 cm, the inner diameter of the, or each conduit (Di) is 0.05 to 1.2 cm, and the ratio of Di/di is 0.008 to 0.4.

According to one embodiment, the device includes more than one ground electrode, the spacing along the length of the conduit between the electrode connected to the positive terminal of the power supply and the ground electrode closest thereto (d1) is 8 to 10 cm, the inner diameter of the, or each conduit (Di) is 0.05 to 1.2 cm, and the ratio of Di/di is 0.005 to 0.15.

According to one embodiment, the device includes more than one ground electrode, the spacing along the length of the conduit between the electrode connected to the positive terminal of the power supply and the ground electrode furthest therefrom (d2) is around 4 cm, the inner diameter of the, or each conduit (Di) is 0.05 to 1.2 cm, there is a spacing between the one of the first and second electrodes closest to the outlet and the outlet of 0.5 cm or more and the ratio of Di/d2 is 0.013 to 0.3.

According to one embodiment, the device includes more than one ground electrode, the spacing along the length of the conduit between the electrode connected to the positive terminal of the power supply and the ground electrode furthest therefrom (d2) is 10 to 20 cm, the inner diameter of the, or each conduit (Di) is 0.05 to 1.2 cm, there is a spacing of at least 0.5 cm between the ground electrode furthest from the electrode connected to the positive terminal of the power supply and the exit and the ratio of Di/d2 is 0.003 to 0.12.

According to one embodiment, the device includes more than one ground electrode, the spacing along the length of the conduit between the electrode connected to the positive terminal of the power supply and the ground electrode furthest therefrom (d2) is 50 to 100 cm, the inner diameter of the, or each conduit (Di) is 0.05 to 1.2 cm, there is a spacing of at least 0.5 cm between the ground electrode furthest from the electrode connected to the positive terminal of the power supply and the exit and the ratio of Di/d2 is 0.0005 to 0.024.

The ratios are summarised in Table 1, Table 2 and Table 3 below.

TABLE 1
Di/d values for the plasma device with a single ground electrode

| Range | d [cm] | Dᵢ [cm] | Range of Dᵢ/d |
|---|---|---|---|
| Smallest | 2-10 | 0.05 | 0.005-0.025 |
| | | 1.2 | 0.6-0.12 |
| Typical | 15-20 | 0.05 | 0.0025-0.0033 |
| | | 1.2 | 0.06-0.08 |
| Largest | 50-100 | 0.05 | 0.0005-0.001 |
| | | 1.2 | 0.012-0.024 |

TABLE 2
Di/d₁ values for the plasma device with
more than one ground electrode.

| Range | d₁ [cm] | Dᵢ [cm] | Range of Dᵢ/d₁ |
|---|---|---|---|
| Smallest | 2 | 0.05 | 0.025 |
| | | 1.2 | 0.6 |
| Typical | 3-6 | 0.05 | 0.008-0.017 |
| | | 1.2 | 0.2-0.4 |
| Largest | 8-10 | 0.05 | 0.005-0.0063 |
| | | 1.2 | 0.12-0.15 |

TABLE 3
Di/d₂ values for the plasma device with
more than one ground electrode.

| Range | d₂ [cm] | Dᵢ [cm] | Range of Dᵢ/d₂ |
|---|---|---|---|
| Smallest | 4 | 0.05 | 0.0125 |
| | | 1.2 | 0.3 |
| Typical | 10-20 | 0.05 | 0.003-0.005 |
| | | 1.2 | 0.06-0.12 |
| Largest | 50-100 | 0.05 | 0.0005-0.001 |
| | | 1.2 | 0.012-0.024 |

Optimising the Di:d ratio optimizes the concentration of hydrogen peroxide generation. The spacing along the length of the conduit between the first (high voltage) electrode and the ground electrode furthest from the first electrode may be surprisingly large for devices including more than one ground electrode, for instance up to 100 cm. According to one embodiment, the spacing between the first (high voltage) electrode and the ground electrode furthest from the first electrode is 8 to 20 cm.

The inter-electrode spacing along the length of the conduit affects the concentration of RONS (such as hydrogen peroxide) and the temperature of the plasma generated.

As well as the ratio of the inner diameter or inner width of the conduit (Di) to the spacing between the first and second electrodes (d), determination of a suitable inter-electrode spacing may be dependent on the electric potential applied between the first and second electrodes, frequency of the power source, the flow rate of the carrier gas, the flow rate of water into the housing, the dimensions of the first and second electrodes, the length of the housing and the conduit (both the total length, the length of the housing between the first (high voltage) electrode and the end of the housing towards the outlet, and the length of the housing between the second (ground) electrode and the end of the housing towards the outlet).

Where the device includes only one ground electrode, the spacing along the length of the conduit between the first and second electrodes is generally less than 30 cm, suitably less than 25 cm, typically less than 20 cm. According to one embodiment, the inter-electrode spacing may be 5 to 30 cm, suitably 7 to 25 cm, generally 15 to 20 cm, typically 16 to 18 cm.

According to one embodiment, the inter-electrode spacing is 5 to 20 cm (generally 10 to 20 cm), the inner diameter or inner width of the or each conduit is 0.5 to 12 mm, the length of the housing between the first (high voltage) electrode and the end of the housing towards the outlet is 11 to 25 cm, where there is a spacing of at least 0.5 cm (generally around 1 cm) between the second (ground) electrode and the end of the housing towards the at least one outlet.

According to one embodiment, the housing has a length, a first end towards the reservoir and a second end towards the outlet;
the ratio of the inner diameter or inner width of the, or each conduit (Di) to the longitudinal spacing between the first and second electrodes (d) is 0.0005 to 0.3;
the length of the housing between the first electrode and the second end is 11 to 25 cm, where there is a spacing of at least 0.5 cm between the second electrode and the second end. The first electrode may be connected to the positive terminal of the power supply, the spacing between the first electrode and the ground electrode furthest therefrom may be 50 to 100 cm and the ratio of the inner diameter or inner width of the or each conduit (Di) to the spacing between the first (high voltage) electrode and the ground electrode furthest from the first electrode may be 0.0005 to 0.024.

Practical discharges at an inter-electrode spacing of more than 50 cm, in particular more than 75 cm such as 100 cm are ordinarily very difficult to operate. The device of the present invention achieves discharges at such inter-electrode spacing, in particular through the inclusion of more than one ground electrode. In addition, the configuration of the housing or conduit can assist in discharges at relatively large inter-electrode spacing. In particular, non-linear housings or conduits are preferred in this regard, for instance helical, coiled or curved housing or conduits, for example conduits undulating along an axis of the plasma device extending in a longitudinal direction (for instance the central longitudinal axis) in a wave form, for instance a sine wave.

According to one embodiment, the device has a relatively large inter-electrode spacing along the length of the conduit. Typically, the inter-electrode spacing is at least 15 cm, typically at least 75 cm, generally around 100 cm. Generally, the first electrode is connected to the positive terminal of the power supply, the second electrode is connected to the negative terminal of the power supply or the ground, and additional ground electrodes are connected to the negative terminal of the power supply or the ground. According to one embodiment the device includes 2, 3, or 4 ground electrodes. Suitably the first electrode is provided within the interstice of the conduit, and the second electrode and any additional ground electrodes are provided externally to the conduit.

Suitably the ratio Di:d is 0.0005 to 0.6, generally 0.01 to 0.6, typically more than 0.05 to 0.6, preferably more than 0.05 to 0.3.

According to one embodiment, the spacing along the length of the conduit between the first electrode and the ground electrode closest to the outlet (d₂) is dependent on the inner diameter or inner width of the, or each conduit (Di). Suitably the ratio of the inner diameter or inner width of the or each conduit (Di) to the spacing between the first electrode and the ground electrode closest to the outlet (d2) is at least 0.001, generally at least 0.002 to 0.2, typically 0.0005 to 0.3. According to one embodiment, the Di/d2 ratio is 0.003 to 0.12.

According to one embodiment, the device has an inter-electrode spacing along the length of the conduit of at least 15 cm, typically at least 75 cm, generally 90 to 110 cm, suitably around 100 to 110 cm. Generally the device includes the first electrode connected to the positive terminal of the power supply, the second electrode connected to the negative terminal of the power supply or the ground, and additional ground electrodes connected to the negative terminal of the power supply or the ground. The inter-electrode spacing is measured from the end of the first electrode to the second electrode, which is the ground electrode closest to the outlet (d2).

Suitably the ratio Di:d2 is 0.0005 to 0.3, generally 0.001 to 0.12, typically 0.005 to 0.06.

According to one embodiment, the housing may taper along its the length, resulting in a substantially conical configuration. The housing may taper towards the end including the outlet for the plasma and the reactive oxygen nitrogen species formed. Suitably, the housing tapers along its length by around 30% to 50%, suitably 30 to 40%. Generally the diameter or width of the housing at the end furthest from the outlet is at least 30% greater than the diameter or width of the housing at the end towards the outlet.

Regardless of the shape of the housing, the inner diameter or width of each conduit is generally substantially constant along its length. Generally the inner diameter or inner width of each conduit is from 0.5 mm to 12 mm.

Suitably the inner width or diameter of all conduits is approximately the same.

The applied voltage between the first and second electrodes may be 0.5 to 30 kV peak to peak (p-p), generally 1 to 20 kV p-p. The power supply may have a low frequency (DC power supply), medium frequency (for instance 1 kHz-1000 kHz), or high frequency (for instance, radio wave frequency 1MHz-30 MHz, micro wave frequency: 2-10 GHz). The medium frequency source could be an alternating current power supply or a pulsed direct current power supply, both of which could generate repetitive waveforms called pulse trains. In some configurations, the plasma source could be configured to operate in continuous or dimming mode. When operated in dimming mode, the pulse duration could vary from 1 to 500 ms and the duty cycle could vary from 5% to 98%.

The flow rate of the carrier gas into the housing may be 0.1 to 15 litres per minute, in particular where the inner diameter or inner width of the or each conduit is from 0.5 to 12 mm. According to one embodiment, the flow rate is 0.5 to 2 litres per minute where the inner diameter or inner width of the, or each conduit is 1.5 mm and the conduit is in the form of a quartz tube. The carrier gas generally includes one or more of air, nitrogen, argon and helium; and typically water vapour.

According to one embodiment, there is provided the device described herein, wherein:
the first and second electrodes are provided within the housing, preferably where all electrodes are provided within the housing;
the ratio of the inner diameter or inner width of the, or each conduit (Di) to the spacing between the first and second electrodes along the length of the conduit (d) is 0.0005 to 0.6, preferably 0.0025 to 0.08.

Typically, the electrode connected to the positive terminal of the power supply is provided within the conduit, and the or all of the electrodes connected to the negative terminal of the power supply is/are provided externally to the conduit, suitably abutting the outer walls of the conduit.

Suitably the device includes a second and optionally a third electrode connected to the negative terminal of the power supply or the ground, wherein the first electrode is connected to the positive terminal of the power supply and suitably wherein the ratio of the inner diameter or inner width of the or each conduit (Di) to the spacing between the first (high voltage) electrode and the ground electrode furthest from the first electrode (d₂) is 0.0005 to 0.3, preferably 0.003 to 0.12. According to one embodiment, the ratio of the inner diameter or inner width of the or each conduit (Di) to the spacing along the length of the conduit between the first (high voltage) electrode and the ground electrode closest thereto (d1) is 0.005 to 0.6, preferably 0.0083 to 0.4.

Generally the housing tapers by at least 30% along its length towards the end including the outlet.

According to one embodiment, there is provided the device described herein, wherein:
the housing has a length, a first end towards the reservoir and a second end towards the outlet;
the ratio of the inner diameter or inner width of the, or each conduit (Di) to the spacing along the length of the conduit between the first and second electrodes (d) is 0.0025 to 0.09, generally 0.003 to 0.08;
the length of the housing between the first electrode and the second end is 11 to 25 cm, where there is a spacing of at least 0.5 cm between the second electrode and the second end.

The inner diameter or the inner width of the, or each conduit may be 1 to 5 mm, and the first and second electrodes may be spaced by 5 to 20 cm.

Where more than one ground electrode is provided, the inter-electrode spacings provided above are the spacing between the first electrode and the ground electrode closest thereto. For the dimensions and parameters detailed above the concentration of RONS (in particular, hydrogen peroxide) generated was found to be maximum at an inter-electrode separation of 16 to 18 cm.

In such embodiments, the width and thickness of the first and second electrodes may be less than 2 cm, the first electrode may have a width of 0.5 to 3 mm, and a thickness of 1 to 3 mm (suitably in the form of a needle inserted inside the conduit (for instance a dielectric tube) and in fluid communication with the reservoir, for instance a stainless steel needle), the second electrode may have a width of 0.3 to 1 cm, generally around 0.4 cm and a thickness of 0.05 to 0.5 cm.

According to one embodiment, the inter-electrode spacing between the end of the first electrode (for instance high voltage needle electrode (stainless steel needle)) and the second electrode (ground electrode (for instance having an associated width 0.4 cm, thickness 0.1 cm)) may be 5 to 20 cm, generally 10 to 20 cm, typically around 17 cm. In such embodiments, the housing may have a length of 18 to 19 cm (typically around 184 mm) between the first (high voltage) electrode and the end of the housing towards the at least one outlet. There is generally a spacing of at least 0.5 cm (generally around 1 cm) between the second (ground) electrode and the end of the housing towards the at least one outlet. The housing may be in the form of a quartz tube having an inner diameter of 1 to 2 mm, generally around 1.5 mm and an outer diameter of 2 to 5 mm, generally around 3 mm.

The range of applied voltage may be 0.5 kV to 30 kV peak-peak (p-p). The excitation source may have a low frequency (DC) power supply, a medium frequency power supply (1 kHz-1000 kHz), or a high frequency power supply (radio wave frequency 1MHz-30 MHz, micro wave frequency: 2-10 GHz)

The carrier gas generally includes water molecules and the housing does not generally have an inlet for water or air. However, air is normally present in housing. The carrier gas flow rate depends on the inner diameter or inner width of the conduit(s). Typically the flow rate may be 0.1 to 15 litres per minute (LPM), suitably 1 to 1.5 LPM, generally around 1.2 litres per minute (LPM). Such flow rates are particularly suitable for use in configurations where the inner diameter or inner width of the conduit is 0.5 to 12 mm.

The plasma device described herein could be utilized for making hydrogen peroxide in plasma activated water and the concentration at a 17 cm inter-electrode gap distance has been found to be maximum (˜20 mM) at a plasma exposure time of 5 minutes in 350 μL of deionized water.

The first electrode is generally a high voltage electrode, that is an electrode connected to the positive terminal of the power supply. The second electrode is generally a ground electrode, that is an electrode connected to the negative terminal of the power supply.

According to one aspect of the present invention, there is provided a plasma device for the generation of RONS, such as hydrogen peroxide, comprising:
a reservoir containing a carrier gas;
a housing in fluid communication with the reservoir, wherein the reservoir includes air or water, or the housing includes an air or water inlet, said housing comprising one or more conduits each formed from a dielectric material, the one or more conduits having an associated inner diameter or inner width;
first (generally high voltage) and second (generally ground) electrodes spaced along the one or more conduits wherein a plasma generation zone is provided in the spacing between the first and second electrodes;
a power supply suitable to apply an electrical potential between the first and second electrodes sufficient to form a plasma through the ionisation of the carrier gas, and to form RONS, such as hydrogen peroxide, from the water molecules and/or from the air;
at least one outlet for the plasma and the RONS such as hydrogen peroxide formed;
wherein the ratio of the inner diameter or inner width of the or each conduit to the longitudinal spacing between the first and second electrodes is 0.0004 to 0.7, generally 0.0005 to 0.6.

According to one embodiment, the inner diameter or inner width of the, or each conduit is 0.05 to 1.5 cm, generally 0.1 to 0.5 cm. Suitably the length of the housing between the first (high voltage) electrode and the end of the housing towards the at least one outlet is 11 to 25 cm. Typically, there is a spacing of 5 to 20 cm along the length of the conduit between the first (high voltage) electrode and the second (ground) electrode. Generally there is a spacing of 0.5 to 5 cm, suitably of 0.5 to 2 cm along the length of the conduit between the second (ground) electrode and the end of the housing towards the at least one outlet.

According to one embodiment, the housing may be formed from a dielectric material, and the conduits may be hollowed out from this.

According to one embodiment, the plasma device may include more than one ground electrode, suitably more than two ground electrodes. This embodiment is particularly suitable for applications of plasma for which higher concentration of hydrogen peroxide at relatively low plasma temperatures are required, for instance, plasma hydrogel therapy where the plasma generated should be suitable for contact with a human or animal body, typically for the treatment of wounds. The plasma generated is generally around room temperature. This embodiment is also particularly suitable for the treatment of thermally sensitive materials, for instance those that require de-contamination of micro-organisms.

The inclusion of more than one ground electrode enhances the concentration of RONS such as hydrogen peroxide generated as well as helping to propagate the plasma plume from the outlet. The footprint of the plasma and RONS composition from the outlet is increased accordingly.

The inclusion of more than one ground electrode allows the production of higher concentrations of RONS such as hydrogen peroxide at room temperature. Where more than one ground electrode is provided, the majority of hydrogen peroxide is produced between the first (high voltage) and the ground electrode closest to it.

According to one embodiment, the device includes two or three ground electrodes.

In such embodiments, the spacing along the length of the conduit between the first (high voltage) electrode and the ground electrode furthest therefrom is 4 to 100 cm, suitably at least 10 cm, generally at least 15 cm. The length of the spacing from the first (high voltage) electrode to the ground electrode closest thereto may be 2 to 10 cm generally at least 5 cm, typically at least 7 cm. Suitably there is a spacing of at least 0.5 cm from the ground electrode furthest from the first (high voltage) electrode and the end of the housing towards the at least one outlet.

According to one embodiment, the device includes three or more ground electrodes. This configuration is particularly suitable where the length of the housing is relatively great, for instance more than 15 cm. The inclusion of three or more ground electrodes increases the concentration of hydrogen peroxide. It also allows the plasma generated between the first (high voltage) electrode and the ground electrode closest thereto to be propelled towards the outlet.

Generally, where the device includes more than one ground electrode, the distance along the length of the conduit between the first (high voltage) electrode and the ground electrode closest to it is 2 to 10 cm and the distance along the length of the conduit between the first (high voltage) electrode and the ground electrode furthest from it is 4 to 100 cm. There is generally a spacing of at least 0.5 cm between the ground electrode furthest from the first (high voltage) electrode and the end of the housing towards the at least one outlet.

According to one embodiment, the housing may have a length 18 to 19 cm (typically around 184 mm) between the first (high voltage) electrode and the end of the housing towards the at least one outlet. The housing generally includes a conduit which may be in the form of a dielectric tube. The spacing along the length of the conduit between the first (high voltage) electrode and the ground electrode closest to it may be between 5 and 6 cm (typically around 56 mm). The spacing along the length of the conduit between the ground electrode closest to the first (high voltage) electrode and the next ground electrode is typically 10 to 10.5 cm. The spacing along the length of the conduit between the first (high voltage) electrode and the ground electrode furthest from it is typically 14 to 16 cm, generally around 15 cm.

According to one embodiment, the plasma device includes a first electrode and two ground electrodes, where the spacing along the length of the conduit between the first electrode and the ground electrode closest to it is 3 to 8 cm, generally 4 to 7 cm, typically around 5 cm, and the spacing between the first electrode and the ground electrode furthest from it is 7 to 20 cm, typically 9 to 18 cm, suitably 10 to 16 cm.

According to an aspect of the present invention, there is provided a plasma device for the generation of RONS such as hydrogen peroxide comprising:
a reservoir containing a carrier gas including water molecules;
a housing in fluid communication with the reservoir, wherein the reservoir includes air or water, or the housing includes an air or water inlet, wherein the housing has a length, the housing comprising a first end towards the reservoir and a second end towards the outlet;
said housing comprising one or more conduits each formed from a dielectric material, the one or more conduits having an associated inner diameter or inner width of 0.5 to 5 mm and;
   a first (high voltage) electrode provided within the one or more conduits and more than one ground electrode provided externally to the one or more conduits, generally abutting the outer surfaces of the one or more conduits, and spaced along the outer surfaces wherein a plasma generation zone is provided in the spacing between the first (high voltage) electrode and the ground electrode closest to the first (high voltage) electrode, wherein the spacing along the length of the one or more conduits between the first (high voltage) electrode and the ground electrode closest to it is 2 to 7 cm and the spacing along the length of the one or more conduits between the first (high voltage) electrode and the ground electrode furthest from it is 15 to 20 cm;
   a power supply suitable to apply an electrical potential between the first (high voltage) electrode and the ground electrode closest to the first (high voltage) electrode sufficient to form a plasma through the ionisation of the carrier gas, and to form RONS such as hydrogen peroxide through the ionisation of the water molecules and optionally through ionisation of air;
   at least one outlet for the plasma and the RONS (in particular, hydrogen peroxide) formed;
wherein the length of the housing between the first (high voltage) electrode and the end of the housing towards the at least one outlet is 11 to 25 cm, and there is a spacing of at least 0.5 to 2 cm between the ground electrode closest to the at least one outlet and the end of the housing towards the at least one outlet.

The ratio of the inner diameter or inner width of the conduit (Di) and the spacing between first and second electrodes (d) is generally 0.0005 to 0.6, suitably 0.001 to 0.6, typically 0.06 to 0.3.

Generally the device includes two or three ground electrodes, typically three ground electrodes.

The first and second electrodes are generally arranged in parallel.

The power supply may be an alternating current power supply or a pulsed direct current power supply. As noted above, the power supply for the generation of plasma could be: (i) a low frequency source (DC) power supply; (ii) a medium frequency source (1-1000 kHz); or (iii) a high frequency source (radio wave frequency: 1-30 MHz or microwave frequency: 2-10 GHz).

The medium frequency source may be an alternating current power supply or a pulsed direct current power supply, both of which generate repetitive waveforms called pulse trains. In some configurations, the plasma source may be configured to operate in continuous or dimming mode. When operated in dimming mode, the pulse duration could vary from 1 to 500 ms and the duty cycle could vary from 5% to 98%. Operating the plasma in dimming mode will reduce the heat of the plasma jet and the target could be treated over a longer duration without rastering.

According to one embodiment, there is provided the device described herein, wherein
the housing has an associated length, wherein a first end of the housing is provided towards the reservoir and a second end of the housing is provided towards the outlet, the power supply has a positive terminal and a negative terminal, the positive terminal being connected to the first electrode and the negative terminal being connected to the second electrode and a third electrode,
the ratio of the inner diameter or inner width of the conduit (Di) and the spacing along the length of the conduit between first and second electrodes (d) is 0.001 to 0.3, typically 0.06 to 0.3;
the length of the housing between the first electrode and the second end is 15 to 25 cm, the spacing between the first electrode and the third electrode is 8 to 20 cm, where there is a spacing of at least 0.5 cm between the third electrode and the second end of the housing.

Generally, the spacing along the length of the one or more conduits between the first electrode and the second electrode is 2 to 10 cm and the inner diameter or inner width of the conduit is 0.05 to 1.5 cm, typically 0.1 to 0.5 cm.

According to one embodiment, the conduits forms a helix around an axis of the plasma device extending in a longitudinal direction, or undulate along an axis of the plasma device extending in a longitudinal direction in a wave form configuration. The portion of the one or more conduits in which the plasma generation zone is formed extend in a helix around a longitudinal axis of the plasma device or a curve along a longitudinal axis of the plasma device. In particular, the section of the one or more conduits between the first and second electrodes may form a helix around an axis of the plasma device extending in a longitudinal direction. Where the device includes more than one ground electrode, the section of the one or more conduits between the first electrode and the ground electrode closest to it is generally in the form of a helix around an axis of the plasma device extending in a longitudinal direction.

Surprisingly, it has been found that helical/curved conduits allow the generation of plasma and RONS such as hydrogen peroxide at a lower temperature than where the conduits extend along the longitudinal axis of the plasma device. Helical/curved conduits nonetheless allow the generation of relatively high concentrations of RONS such as hydrogen peroxide in a controllable, predictable manner. Additionally, the helical /curved configuration provides the additional advantage of a reduction in the total height of the housing whilst maintaining a greater conduit length than the height of the housing, or a similarly dimensioned arrangement including longitudinally extending conduits.

Equivalently dimensioned housings including longitudinally extending conduits were compared with those including helical or curved conduits. For housings of the same dimensions, including first and second electrodes of the same dimensions and provided at the same inter-electrode separation, and the application of the same electrical potential, the plasma generated was found to be around 5° C. less for the housing including the helical/curved conduits compared to the equivalent housing including conduits extending along the longitudinal axis of the device.

According to one embodiment, the housing may include one or more conduits in the form of one or more dielectric tubes (such as quartz, glass, PTFE, alumina, etc.), where the conduits may form a helix around the longitudinal axis of the device. The first electrode may be connected to a high voltage power supply and the second electrode may be connected to the ground. The high voltage electrode may be in the form of a metallic needle electrode (such as stainless steel, tungsten, silver, copper, etc.) in fluid communication with the reservoir for the carrier gas. The carrier gas typically includes water molecules.

Although the first and second electrodes are provided within the housing, according to one embodiment, the second (ground) electrode may be provided around the conduit. The second electrode may form a helix around an outside surface of the conduit. According to one embodiment, the second, ground electrode may be wrapped around the outside of the conduit which is in the form of a dielectric tube. The second, ground electrode may be formed of a conductive material. Suitable materials would be known to the skilled man. Mention may be made of copper in this regard.

According to one embodiment, the first and second electrodes may be provided around the outside of the conduit or each conduit. In such embodiments, an outside surface of the conduit(s) is formed of a dielectric material. The first and second electrodes are nonetheless provided within the housing.

Suitably, the first electrode may be inserted into the conduit formed from dielectric material and may be in fluid communication with the reservoir. The second electrode may be wrapped around an outside surface of the conduit.

According to one aspect of the present invention, there is provided a plasma device for the generation of RONS, such as hydrogen peroxide comprising:
a reservoir containing a carrier gas;
a housing in fluid communication with the reservoir, wherein the reservoir includes air or water, or the housing includes an air or water inlet, said housing comprising one or more conduits each formed from a dielectric material;
first and second electrodes spaced along the conduit wherein a plasma generation zone is provided in the spacing along the length of the conduit between the first and second electrodes, wherein the conduit(s) form a helix around the longitudinal axis of the plasma device;
a power supply suitable to apply an electrical potential between the first and second electrodes sufficient to form a plasma through the ionisation of the carrier gas, and to form RONS such as hydrogen peroxide from the water molecules and optionally through ionisation of air;
at least one outlet for the plasma and the RONS such as hydrogen peroxide formed.

Generally the ratio of the inner diameter or inner width of the or each conduit to the spacing between the first and second electrodes in 0.0005 to 0.6.

According to one embodiment, the housing includes more than one conduit, the first electrode is provided within the conduit, and the second electrode is provided around an outer surface of the conduit wherein the plasma generation zone is provided within the more than one conduit in the longitudinal spacing between the first and second electrodes. In such an embodiment, the plasma device includes more than one outlet for the plasma and the RONS such as hydrogen peroxide formed.

The provision of more than one plasma generation zone results in more RONS (generally including hydrogen peroxide) being generated, in particular the plasma includes a higher concentration of RONS, in particular hydrogen peroxide for a similar flow rate of carrier gas and water molecules.

Generally some or all of the conduits extend in a longitudinal direction along the housing, along or parallel to the longitudinal axis of the housing.

According to one embodiment, the housing may taper along its length resulting in a substantially conical configuration, and some or all of the conduits may extend towards the central longitudinal axis of the housing at the end of the housing towards the outlet.

Alternatively or additionally, some or all of the conduits form a helix around an axis of the housing extending in a longitudinal direction. Alternatively or additionally, some or all of the conduits undulate along an axis of the housing extending in a longitudinal direction in a wave form such as in the form of a sine wave.

The housing may comprise one to ten of the conduits, suitably one to seven, typically, 3 to 5 conduits.

Where the housing includes a single conduit in which the plasma generation zone is provided, the conduit generally extends along the longitudinal axis of the housing.

Alternatively, the housing may include a single conduit in which the plasma generation zone is provided, and the conduit may form a helix around the longitudinal axis of the housing.

According to one embodiment, the plasma device includes one to ten conduits extending in a longitudinal direction along the housing, along or parallel to the longitudinal axis of the housing; suitably three to seven; typically four to six.

According to one embodiment, the plasma device includes one to fifteen (typically one to thirteen) conduits, forming a helix around an axis of the housing extending in a longitudinal direction; suitably three to five; alternatively, six to eight.

According to a further embodiment, the plasma device may include one or more conduits extending in a longitudinal direction along the housing, along or parallel to the longitudinal axis of the housing, and one or more conduits forming a helix around an axis of the housing extending in a longitudinal direction; suitably one to nine conduits extending in a longitudinal direction along the housing and one to nine conduits forming a helix around an axis of the housing extending in a longitudinal direction.

Suitably the plasma generation zone has an associated minimum volume of 0.0001 to —35 cm³.

The volume of the plasma generation zone is dependent on the sizing of the interstice provided within the walls of the conduits, the inter-electrode spacing along the length of the conduit, and the number of conduits. Where the housing includes a single conduit with an associated inner diameter or inner width of 0.05 to 0.5 cm and an inter-electrode spacing of 2 to 20 cm, the minimum volume of the plasma generation zone is 0.001 to 5 cm³, generally 0.005 to 4 cm³. Where the housing includes a single conduit with an associated inner diameter or inner width of more than 0.5 to 1.2 cm, the minimum volume of the plasma generation zone is 4 to 25 cm³. Where the housing includes seven conduits with an associated inner diameter or inner width of 0.5 to 5 mm and an inter-electrode spacing of 2 to 20 cm, the minimum volume of the plasma generation zone is 0.5 to 27 cm³, generally 0.05 to 21 cm³.

Where the housing includes nine conduits with an associated inner diameter or inner width of 0.5 to 5 mm and an inter-electrode spacing of 2 to 20 cm, the minimum volume of the plasma generation zone is 0.6 to35 cm³, generally 0.7 to 30 cm³.

The associated results are summarised in Table 4 below

TABLE 4
Minimum volume of the plasma devices with 1,
7 and 9 conduits for d or d₂ up to 20 cm.

| | Dᵢ | d or d₂ | Minimum volume range | | |
|---|---|---|---|---|---|
| Range | [cm] | [cm] | 1 - jet [cm3] | 7 - jet [cm3] | 1 - jet [cm3] |
| Smallest | 0.05-0.5 | 4-20 | 0.008-0.04 | 0.055-0.27 | 0.07-0.35 |
| Typical | 0.15-0.5 | 4-20 | 0.07-3.93 | 0.49-27.49 | 0.64-35.34 |
| Largest | 0.5-1.2 | 4-20 | 4.52-22.62 | 31.67-158.33 | 40.71-203.57 |

According to one aspect of the present invention, there is provided a plasma device for the generation of reactive oxygen nitrogen species (RONS) comprising:
a reservoir containing a carrier gas;
a housing in fluid communication with the reservoir, wherein the reservoir includes air or water, or the housing includes an air or water inlet, said housing comprising more than one conduit, each formed from a dielectric material,
first and second electrodes spaced along the one or more conduits wherein a plasma generation zone is provided in the spacing along the length of the one or more conduits between the first and second electrodes;
a power supply suitable to apply an electrical potential between the first and second electrodes sufficient to form a plasma through the ionisation of the carrier gas, and to form RONS such as hydrogen peroxide through the ionisation of the water molecules and optionally air;
more than one outlet for the plasma and the RONS formed.

Typically, the plasma device includes one to fifteen conduits, wherein six to eight of the conduits form a helix around the longitudinal axis of the device.

The, or each conduit may be in the form of a tube. Generally each of the conduits may be in the form of dielectric tube, typically a glass tube or a quartz tube.

The plasma device of the present teaching includes at least one outlet for the plasma and the RONS such as hydrogen peroxide formed. As the carrier gas is generally ionised in the spacing between the first and second electrodes, the RONS such as hydrogen peroxide formed with other plasma species (including electrons, ions, ultra-violets, reactive species, etc.) are generally mixed together upon formation, and so the outlet or each outlet typically releases a mixture of different plasma species and RONS such as hydrogen peroxide.

Generally the plasma device includes more than one outlet, suitably five to twenty outlets, typically ten to fifteen outlets. Including a greater number of outlets may maximise the area of delivery of plasma and RONS such as hydrogen peroxide, increasing the “footprint” of delivery and reducing the risk of contacting human tissue with high concentrations of reactive oxygen species, reducing the risk of inadvertent excess heating and reducing the risk of dehydrating the target. The temperature of the plasma composition generated is a generally reduced compared to standard plasma compositions which is also beneficial for use on the human body, as well as being beneficial for use on other thermally sensitive materials. Suitably the device includes a flowmeter configured to control the flow of carrier gas from the reservoir to the housing. The device may include more than one flowmeter, suitably the device includes a second flowmeter configured to control the flow of air or water through the inlet. Suitable flowmeters will be known to one skilled in the art. The flowmeter may be in the form of a mass flow controller or rotameter.

The carrier gas typically comprises a nobel gas. The carrier gas may be selected from the group consisting of one or more of helium, argon, nitrogen, hydrogen, krypton, neon, and xenon. Typically the carrier gas includes at least 90 vol. % of one or more of helium, argon, nitrogen, hydrogen, krypton, neon, and xenon, generally at least 95 vol. %, typically at least 99 vol. %. Suitably the carrier gas also includes water molecules. Generally the carrier gas consists of or comprises argon, typically the carrier gas comprises argon and water.

Where the carrier gas includes water, water is present at an amount of less than 1 vol. %, generally 0.00005 to 0.005 vol. %, generally 0.001 to 0.005 vol. % water. As noted above, where the device includes an air or water inlet, the carrier gas may be mixed with water or air following introduction to the housing. In such embodiments, the carrier gas may be mixed with up to 10 vol. % water, generally 1 to 5 vol. % water. In addition, some ambient humidity is present in the device from the ambient environment. The greater the volume within the housing, the more ambient moisture will be present and will be mixed with the carrier gas. The amount of water in the carrier gas should not exceed 10 vol. % as this will affect the propensity of the carrier gas to form a plasma.

According to one embodiment, the carrier gas includes 99.999 vol. % argon and around 0.00005 vol. % water.

The reservoir containing the carrier gas is generally pressurised at a pressure greater than atmospheric pressure. The reservoir may be at a pressure of around 100 to 300 bar.

The housing may include an air or water inlet, generally a water inlet. Such an inlet may provide access to the surrounding environment, or may provide access to a specified air or water reservoir which may be under pressure and/or may include a pump. Such an embodiment allows the introduction of water molecules into the housing where the carrier gas does not include water molecules. Where present, the air or water inlet is generally provided between the reservoir and the first electrode to allow mixing of the carrier gas and the air or water prior to delivery to the plasma generation zone, and thus ensuring simultaneous ionisation of the carrier gas and water molecules.

Alternatively, or additionally the carrier gas includes water molecules.

According to one embodiment, ambient water molecules in air may also be ionised in the plasma generation zone to form RONS including hydrogen peroxide, and other derivatives of oxygen/nitrogen (ozone, nitrous acid, peroxynitrate, etc.).

According to one embodiment, the device may include a downstream inlet provided between the second electrode and the outlet. The downstream inlet may allow the introduction of gases into the housing, said gases including one or more of those selected from the group consisting of (a) carrier gas (b) O₂ (c) N₂ (d) H₂O, or a combination of one or more of (a), (b), (c) and (d). The introduction of such gases may control the formation of reactive species in particular RONS. For example, to maximise the formation of hydrogen peroxide, the carrier gas may include water molecules, and carrier gas including water molecules may also be introduced through the downstream inlet. The gas introduced through the downstream inlet may be ionised to enhance the concentration of H₂O₂ in the downstream region or may reduce the formation of RONS other than hydrogen peroxide.

The first and second electrodes are generally formed from materials having a conductivity of at least 1.4 MS/m at 20° C., typically at least 1.45 MS/m at 20° C. They may be made of materials such as copper, aluminium, silver, stainless steel. However, the skilled man would be well aware of other examples of suitable materials.

The power supply may include an electrical connector connected to the electrodes. Generally the positive terminal of the power supply is connected to the first electrode, and the negative terminal of the power supply is connected to the second electrode (and/any other ground electrodes provided).

According to one embodiment, the device includes a surrounding gas, provided between the electrode closest to the at least one outlet and the end of the housing towards the at least one outlet wherein the surrounding gas is selected from the group consisting of argon, helium, nitrogen, oxygen and mixtures thereof. The provision of the surrounding gas may limit the interaction of the plasma with ambient air molecules.

Method

According to a further aspect of the present invention, there is provided a method of forming a plasma including reactive oxygen nitrogen species (RONS), in particular hydrogen peroxide comprising:
providing the device as described herein;
providing a flow of the carrier gas and air or water through the housing;
applying an electrical potential between the first and second electrodes to ionise the carrier gas to form a plasma and to ionise oxygen and hydrogen in the air or in the water to form RONS such as hydrogen peroxide.

According to an aspect of the present invention there is provided a method of forming a plasma including RONS such as hydrogen peroxide comprising:
providing the device as described herein;
providing a flow of the carrier gas including water molecules through the housing;
applying an electrical potential between the first and second electrodes to ionise the carrier gas including water molecules to form a plasma and RONS such as hydrogen peroxide and optionally to ionise ambient air molecules to RONS.

The method of the present invention allows the controllable, predictable generation of RONS such as hydrogen peroxide at temperatures suitable for application to human tissue, or other heat sensitive material. The application of the electrical potential acts to generate the plasma from the ionisation of the carrier gas by electrical discharge in the housing. Generally RONS such as hydrogen peroxide are formed through the ionisation of water molecules (including water molecules in air). Water molecules can be dissociated to form hydroxyl radicals which subsequently combine to form hydrogen peroxide. Optionally, air molecules may also be ionised to form RONS.

The rate of generation of RONS such as hydrogen peroxide can be readily controlled through control of one or more flow rate of, for example, the carrier gas (generally including water molecules) through the housing, flow rate of water molecules through the housing, the presence and/or flow rate of air through the housing, the dimensions of the housing, the dimensions and number of conduits, the inter-electrode spacing as detailed above, and the applied voltage from the power supply.

According to one embodiment, the method includes providing a surrounding gas between the electrode closest to the at least one outlet and the end of the housing towards the at least one outlet wherein the surrounding gas is selected from the group consisting of argon, helium, nitrogen, oxygen and mixtures thereof. The provision of the surrounding gas may limit the interaction of the plasma with ambient air molecules.

The device and method of the present invention allows the high concentration delivery of RONS such as hydrogen peroxide at relatively low temperatures over a wide area. The present invention allows production and delivery of sufficient/high concentration of RONS such as hydrogen peroxide and nitrous acid over relatively large surface areas whilst achieving control over the (“delivered”) plasma-gas temperature. This allows the decontamination or treatment of thermally sensitive materials, whilst avoiding damage or dehydration of the thermally sensitive materials.

The methods of the present invention may be used to activate hydrogel dressings loaded with therapeutic agents. Alternatively, the plasma generated according to the invention may be used to decontaminate surfaces of micro-organisms and/or parasites directly. The methods of the present invention allow the delivery of RONS such as hydrogen peroxide at sufficient concentration and over a sufficient area to allow such decontamination on a clinically acceptable time scale.

The methods of the present invention also allow the delivery of RONS such as hydrogen peroxide directly to thermally-sensitive materials allowing decontaminate micro-organisms and parasites.

The plasma produced according to the methods of the present invention may have a temperature of less than 40° C. ensuring minimal denaturing of proteins.

The plasma may include RONS such as one or more of hydrogen peroxide, ozone, nitrous acid and peroxynitrate. These RONS may be generated through the ionisation of water and/or air in the plasma generation zone within the housing.

The concentration of RONS generated according to the present invention may be tailored according to the required use, for instance the area of the surface to be treated, the volume of the body to be treated, limits on plasma exposure time.

The concentration depends on a variety of controllable parameters, such as the inter-electrode spacing, the volume of the plasma generation zone, the electrical potential applied, and the carrier gas flow rate.

By way of example, a device of the present invention generates hydrogen peroxide having a concentration of around 10 μM to 30 mM, typically around 20 mM hydrogen peroxide where the device includes a single conduit, an inter-electrode spacing of around 17 cm, a carrier gas flow rate of around 1.2 SLPM, where the carrier gas includes 99.999 vol % argon and 0.00005 vol. % water; wherein the electrical potential is around 10 kV p-p and has an associated frequency of around 23.5 kHz. In contrast, a formulation generated by an equivalent multi-jet device of the present invention including more than 5 conduits (generally 9 to 15 conduits) may have a hydrogen peroxide concentration of from 1-1000 mM.

A plasma formulation including around 20 mM hydrogen peroxide would be suitable to treat a target liquid having a volume of around 350 μL for around 5 minutes.

The flow rate of carrier gas and water molecules through the housing is generally limited to the laminar mode, in particular the gas flow inside the housing should not exceed the laminar mode. For a particular carrier gas, this could be estimated through the calculation of Reynold's number. For a quartz tube of 1.5 mm inner diameter, this value is in the range of 0.2 to 2 litres per minute, suitably 0.8 to 1.2 litres per minute, typically 1 to 1.5 litres per minute.

The electrical potential is generally applied at a voltage of 2 to 15 kV, generally at a power range of 0.5 to 10 Watts and typically at a frequency 5 to 60 kHz. The electrical potential may be applied AC. Alternatively the electrical potential may be applied as pulsed DC. Where the air or water molecules are introduced to the housing separately to the carrier gas, the air and/or water molecules are generally at atmospheric pressure.

Atmospheric air from the surrounding environment may come in contact with the plasma at in the housing leading to the formation of RONS for instance, hydrogen peroxide, excited nitrogen, nitrates, nitrites, peroxynitrates, atomic oxygen, etc.

The plasma and RONS such as hydrogen peroxide generation may also be controlled by controlling the volume of the plasma generation zone, the number of conduits in the plasma device including plasma generation zones and the inter electrode separation.

The plasma and RONS such as hydrogen peroxide generally flows through the outlet(s) to contact a gelled or liquid surface (for example, a hydrogel dressing) resulting in the formation of RONS such as hydrogen peroxide of desired concentration. The temperature of the plasma composition emitted must be controlled where the target is heat sensitive, for example a human or animal body. Generally, the temperature for the formation of RONS such as hydrogen peroxide in liquids including deionized water or other solutions, could be less than 100° C., typically 40 to 70° C. The temperature of the plasma composition including hydrogen 91peroxide for activation of hydrogel dressings is less than 50° C., typically 30 to 40° C.

According to one embodiment, the plasma composition including RONS such as hydrogen peroxide is emitted through more than one outlet, allowing delivery over an area of around 0.5 cm² to 10 cm², generally 3 to 7 cm². The concentration of RONS such as hydrogen peroxide generated at room temperature would suitably be in the range of 1-1000 mM.

According to one embodiment, there is provided a method of deactivation of micro-organisms on a surface comprising:
generating a plasma comprising RONS such as hydrogen peroxide using the device as described herein;
contacting the surface with the plasma comprising RONS such as hydrogen peroxide.

The plasma may have a temperature of 30 to 40° C., and may be emitted from the device over an area of 0.5 cm² to 10 cm², generally 3 to 7 cm². The concentration of RONS in the plasma is generally from 1 to 1000 mM.

Plasma Treatment Method

According to a further embodiment, there is provided a plasma treatment method for a patient in need thereof comprising:
providing a hydrogel on an anatomical region of interest of the patient;
generating a plasma comprising RONS such as hydrogen peroxide using the device described herein, and/or using the method described herein;
contacting a surface of the hydrogel with the plasma comprising RONS such as hydrogen peroxide; wherein contact of the hydrogel with the plasma activates the hydrogel dressing.

The hydrogel typically includes therapeutic agents, and activation of the hydrogel typically triggers release of the therapeutic agents.

The therapeutic agents can be added directly into the hydrogel, or alternatively may be sequestered within capsules (e.g. vesicles), held in an inert form, or are covalently linked to the gel matrix ready for activation by RONS such as hydrogen peroxide.

Typical therapeutic agents include one or more of anti-microbials, anti-biotics, antiseptics, cancer agents.

The anatomical region of interest may be one or more of a wound, an infected area (for instance by micro-organisms or parasites) and a burn.

The dressing materials employed are non-toxic and biocompatible hydrogels, with controllable rheological properties and water content.

Generally the thickness of the hydrogel dressing is 1 to 50 mm. Optimum thicknesses may be in the range of from 1 to 5 mm, suitably 1 to 2 mm, typically 3-4 mm. Anti-microbials may be incorporated into or onto these hydrogels.

Antimicrobial release from hydrogels will generally be through three mechanisms: (i) reactive oxygen/nitrogen species (RONS) (including hydrogen peroxide) flux release; (ii) CAP (cold atmospheric plasma!) pH-stimulated gel collapse and ‘pump-out’ of antimicrobial; and (iii) CAP RONS activated release (by covalent bond breakage) of antimicrobials grafted to PVA chain.

Typically topical antimicrobials are released from hydrogels in accordance with the method of the present invention. Suitable antimicrobials include chlorhexidine-digluconate; cetrimide; polyhexanide; and poviodone-iodine although the skilled man would be well aware of other antimicrobials suitable for use in the present invention.

The antibiotics that could be covalently grafted to PVA include Triclosan, ciprofloxacin and gentamicin sulphate, for instance, and are all used in clinical care. The mode of chemical linkage may be such that the release of the antibiotics may be triggered by RONS such as H₂O₂ by covalent bond breakage, where linked by, for example, boronic acid chemistry. In this regard, mention may be made of Chemical Communications, 2019 55 (100), 15129-15132 authored by B L Patenall, G T Williams, L Gwynne, L J Stephens, E V Lampard et al., incorporated herein by reference.

These antibiotics tend to be effective only when applied topically, directed at high concentration and contained within the wound site.

Using RONS (including hydrogen peroxide) to trigger antimicrobial delivery into the wound means that a concentration, many times greater than target bacterial minimum inhibitory concentration (MIC), can be released quickly to where it is needed: delivering the right antimicrobial, to the right place at the right time.

Generally the activation of the hydrogel causes release of the therapeutic agents. Activation is through the delivery of RONS such as hydrogen peroxide from the plasma. The challenge overcome by the device and methods of the present teachings is the production of enough RONS such as hydrogen peroxide for activation of the hydrogel without unacceptable heating and dehydration of the hydrogel. In addition, electrical stimulation of human tissue, and contact with reactive oxygen and nitrogen species must be controlled. The design of the device of the present teachings helps to ensure the delivery of therapeutic agents to the anatomical region of interest controllably, predictably and safely. Particular aspects of the device which overcome the challenges associated with plasma treatment methods include the provision of numerous plasma generation zones in the housing, as well as the provision of the numerous outlets for the plasma/H₂O₂ composition. The inclusion of one or more helical housings for the generation of RONS such as hydrogen peroxide may also assist, as may the inclusion of more than one linear or helical conduits, as well as the optimisation of the inter-electrode separation.

The drug is co-delivered with plasma-derived species, specifically RONS such as hydrogen peroxide, and optionally additional reactive oxygen species and nitrogen species.

The temperature of the plasma composition which contacts the hydrogel surface is preferably less than 50° C., suitably 30 to 40° C.

The method may be further optimised by control of the distance between the outlet(s) and the hydrogel surface, generally this is 0.5 to 5 cm, suitably 1 to 2 cm, alternatively 3 to 5 cm.

Further optimisation is achieved through control of the contact time between the hydrogel and the plasma. Suitable contact times are generally 1 to 5 minutes per 5 cm ² area, typically 3 to 4 minutes per 5 cm ² area.

The skilled person will be aware of suitable hydrogels. Mention can be made of poly vinyl alcohol gel, including crosslinked poly-vinyl-alcohol (PVA) such as cryo-crosslinked PVA, and antimicrobial grafted PVA; sodium polyacrylate; and agarose gel. In this regard, mention may be made of Chemical Communications, 2019 55 (100), 15129-151302 authored by B L Patenall, G T Williams, L Gwynne, L J Stephens, E V Lampard et al., incorporated herein by reference.

The plasma treatment methods may be used in the treatment of infections, such as ulcers or wounds, including diabetic foot ulcers and surgical site infections. Other applications can be for instance in the release of cancer agents in the treatment of melanoma.

The patient is generally a mammal. Mention may be made of humans, horses, dogs, cats, camels, cattle.

System According to a further embodiment, there is provided a system including the device as described herein and a hydrogel dressing comprising therapeutic agents wherein the hydrogel dressing is activatable upon contact with a plasma comprising RONS such as hydrogen peroxide to release the therapeutic agents.

Kit

According to a further embodiment there is provided a kit of parts comprising the plasma device described herein and instructions for use. Generally the kit includes one or more hydrogel compositions as described herein.

The kit may include means to measure the flow rate of the carrier gas into the housing and/or means to measure the flow of water molecules into the housing, means to control the electrical potential, means to measure the temperature of the plasma composition emitted from the outlet(s).

Kits can further include instructions for performing the methods described herein and/or interpreting the results, in accordance with any regulatory requirements. In addition, software can be included in the kit for analysing one or more of the flow rate of the carrier gas and/or flow rate of water molecules into the housing, analyzing and controlling the electrical potential, measuring the concentration, flow rate and/or temperature of the plasma composition emitted from the outlet(s), as well as the area over which the plasma composition in dispensed from the outlet(s).

Preferably, the kits are packaged in a container suitable for commercial distribution, sale, and/or use, containing the appropriate labels, for example, labels including the identification of the chemical reactants included.

The present invention will now be described by way of example only with reference to the associated figures in which:

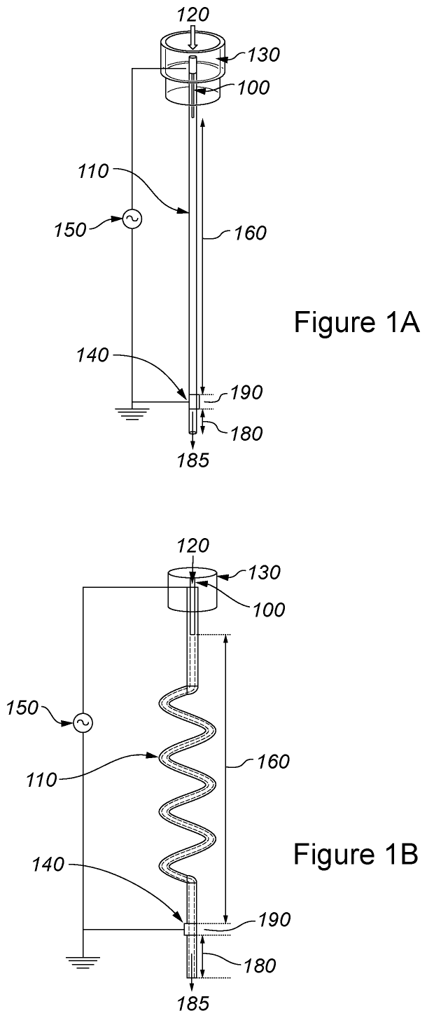

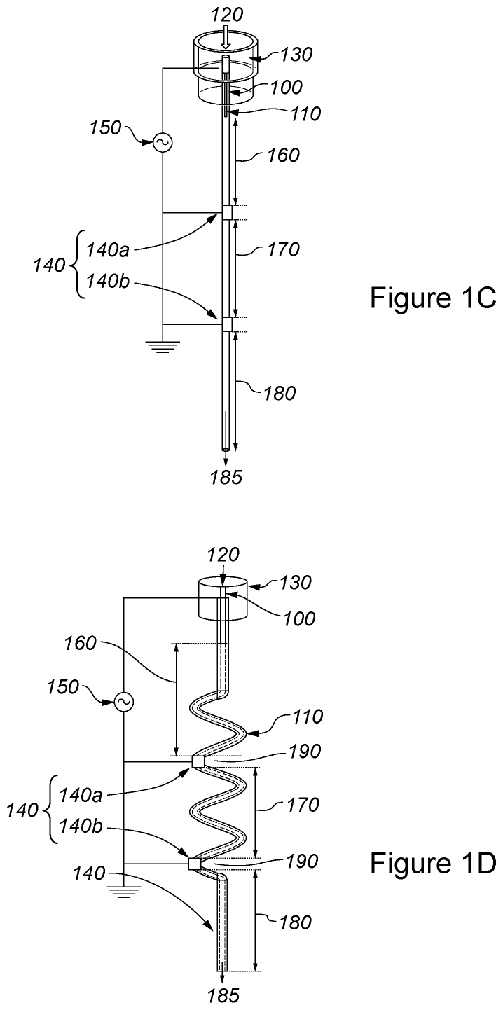

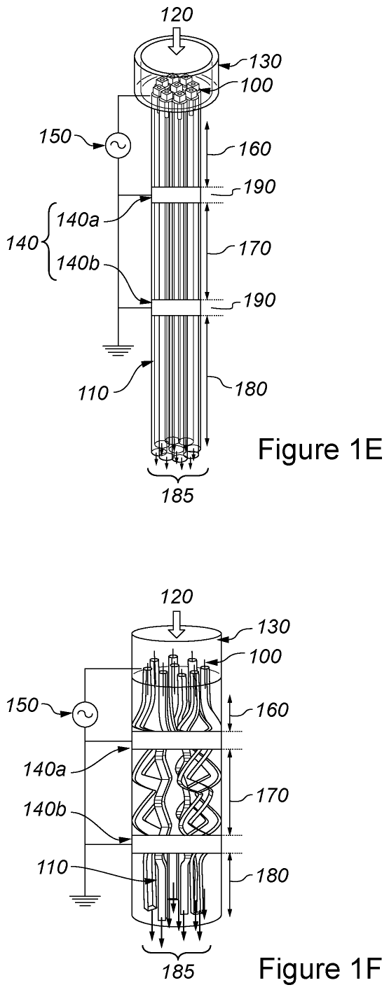

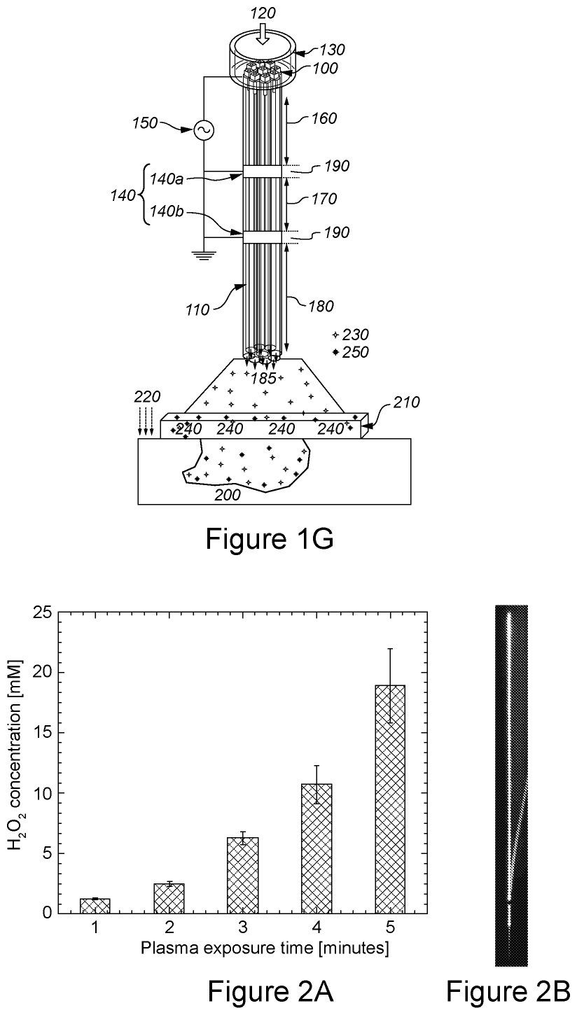

FIG. 1A shows an embodiment of the plasma device of the present invention comprising a housing extending along the longitudinal axis of the device;

FIG. 1B shows an embodiment of the device of the present invention comprising a housing helically extending around the longitudinal axis of the device;

FIG. 1C shows an embodiment of the device of the present invention comprising a housing extending along the longitudinal axis of the device for activating hydrogels and application to patients;

FIG. 1D shows an embodiment of the device of the present invention comprising a housing helically extending around the longitudinal axis of the device for activating hydrogels and application to patients;

FIG. 1E shows an embodiment of the device of the present invention including a plurality of conduits extending along the longitudinal axis of the device to provide a multi-jet device;

FIG. 1F shows an embodiment of the device of the present invention including a plurality of conduits extending helically around the longitudinal axis of the device to provide a multi-jet device;

FIG. 1G shows a schematic representation of the device of 1E used in the activation a hydrogel;

FIG. 2A shows the concentration of hydrogen peroxide produced in accordance with Example 1 in plasma treated deionized water at different plasma exposure time;

FIG. 2B shows a photo of the plasma jet according to the description in Example 1;

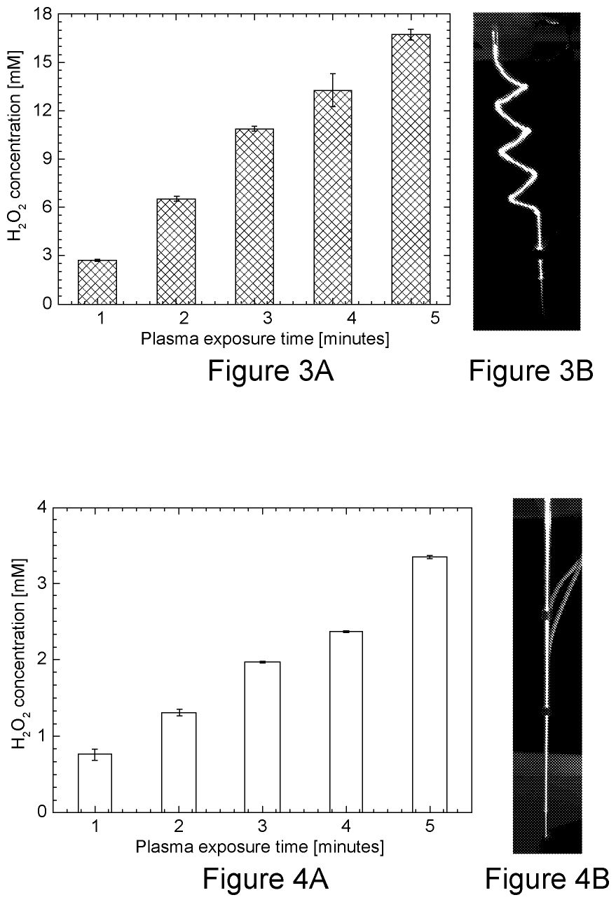

FIG. 3A shows the concentration of hydrogen peroxide as measured from the plasma activated water prepared in accordance with Example 2;

FIG. 3B shows a photo of the plasma jet according to the description in Example 2;

FIG. 4A shows the concentration of hydrogen peroxide produced in plasma treated deionized water at different plasma exposure times in accordance with Example 3;

FIG. 4B shows a photo of the plasma jet according to the description in Example 3;

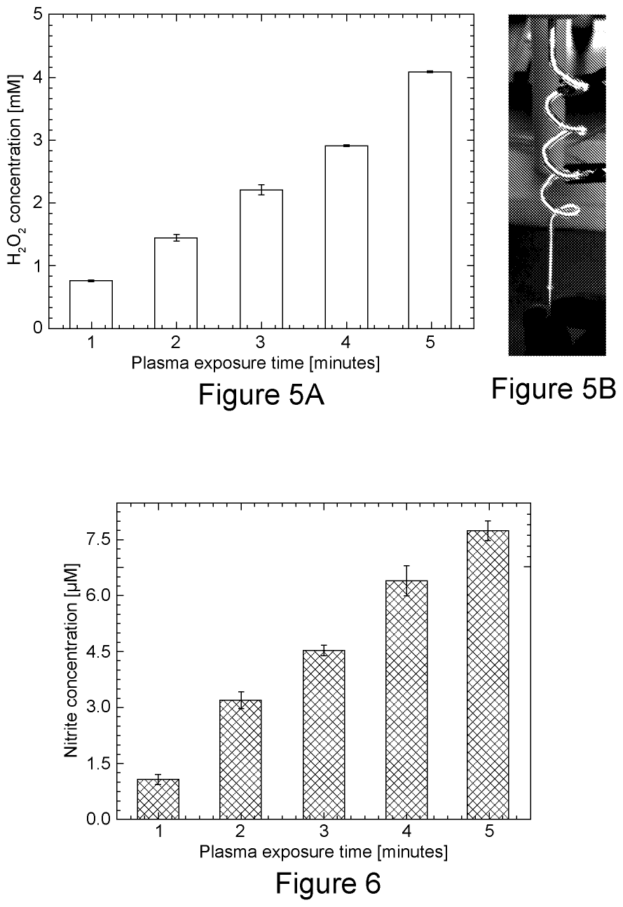

FIG. 5A shows the concentration of hydrogen peroxide produced in plasma treated deionized water at different plasma exposure times in accordance with Example 4;

FIG. 5B shows a photo of the plasma jet according to the description in Example 4;

FIG. 6 shows the concentration of nitrites produced in plasma treated deionized water at different plasma exposure times in accordance with Example 1;

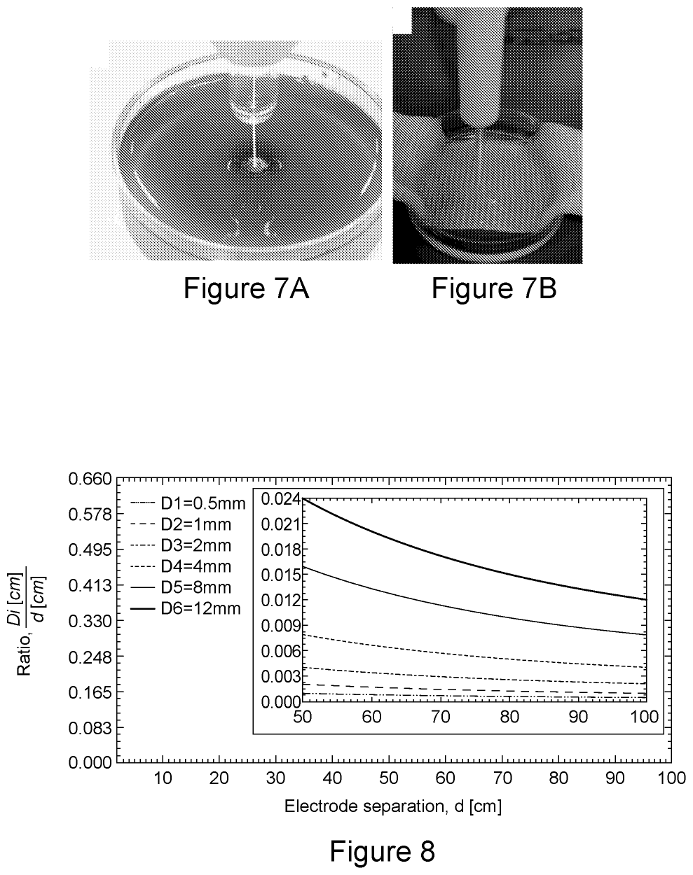

FIG. 7 shows the stability of the hydrogel against continuous plasma exposure time of two minutes in accordance with Example 6. The target with (B) argon plasma jet can tolerate the jet temperature whereas the (A) helium plasma jet burns it;

FIG. 8 shows the relationship between the ratio of the inner diameter of dielectric tubes (Di) and the spacing between first and second electrodes (d) in accordance with Example 7;

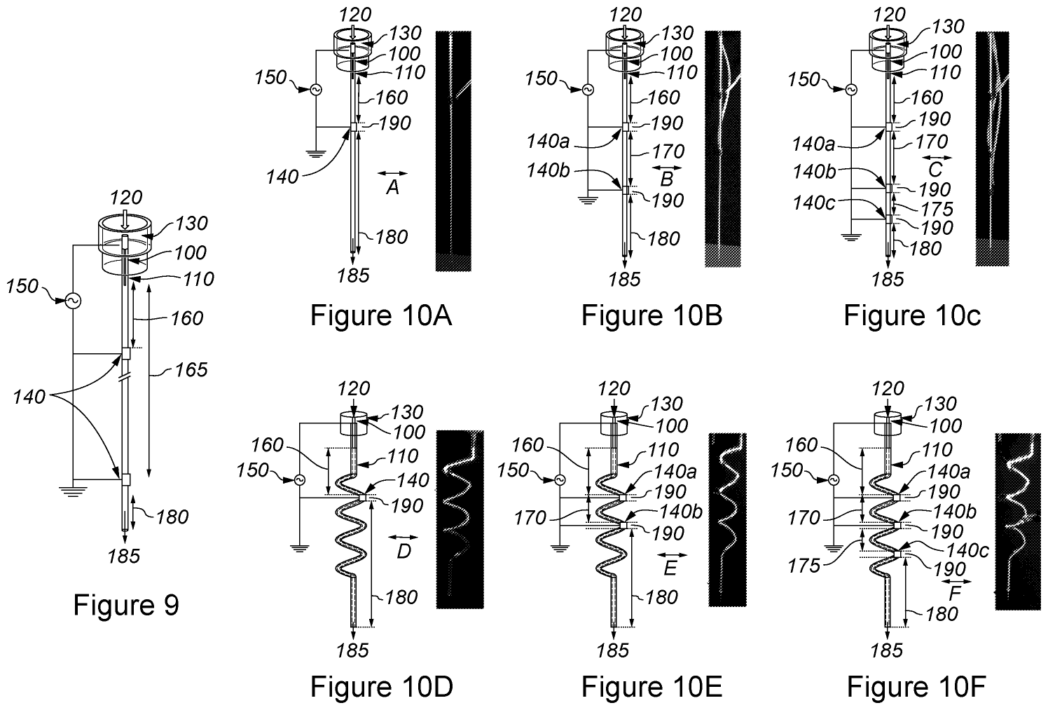

FIG. 9 provides a schematic representation of the device of the present invention with more than one ground electrode in accordance with Example 9 and 10;

FIG. 10 provides schematic representations and photographs of the plasma jets in linear and helical configurations with one (10A and 10D), two (10B and 10E) and three (10C and 10F) ground electrodes in accordance with Example 9;

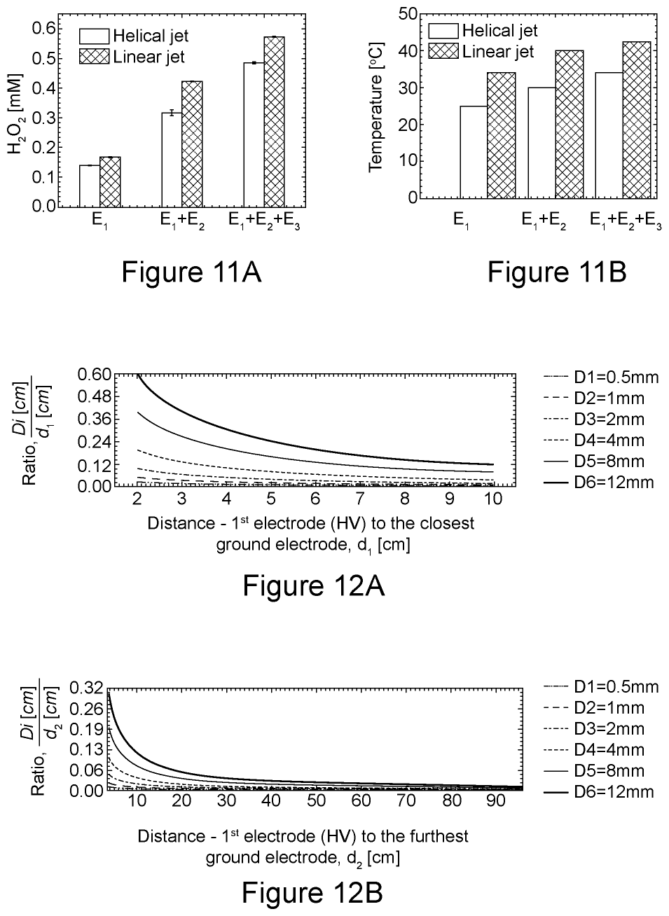

FIG. 11 shows the measurement of the (A) concentrations of the hydrogen peroxide and (B) temperatures of the plasma jets in helical and linear configurations in accordance with Example 9. The plasma jets with one, two and three ground electrodes in both configurations are represented by E₁, E₁+E₂ and E₁+E₂+E₃ respectively;

FIG. 12 shows the dimensions of the plasma jets for one, two and three ground electrodes in terms of ratio Dᵢ/d₁ (FIG. 12A) and Dᵢ/d₂ (FIG. 12B) in accordance with Example 10;

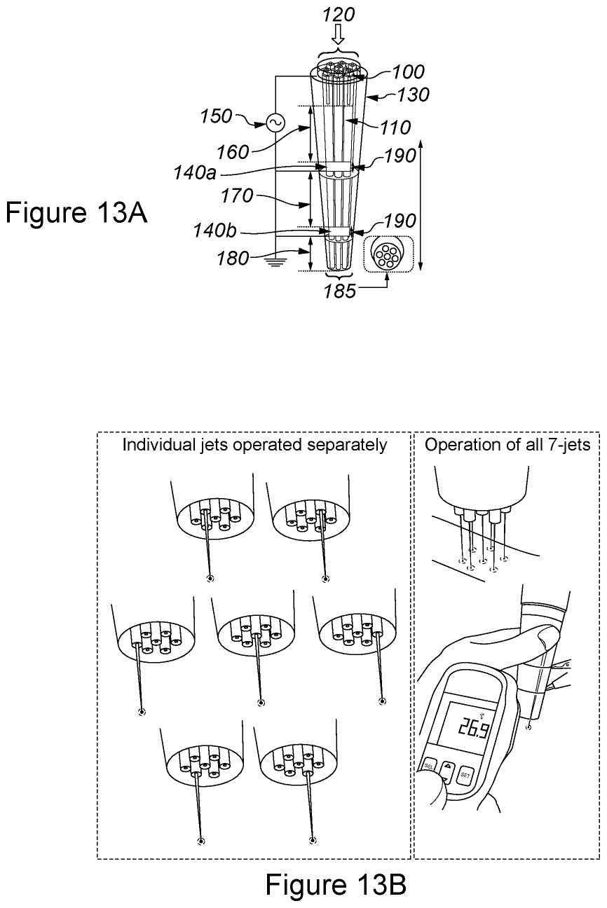

FIG. 13A shows the schematic of the conical design of the multi jet plasma device in accordance with Example 11. FIG. 13B shows the photographs of the corresponding device with seven plasma jets operated individually (left) or together (right). The temperature of the plasma jet (bottom-right) measured with an infrared thermometer is below 30 degree celsius.

In the figures, the following reference numerals are used:
100 First electrode; high voltage electrode; needle electrode
110 Conduit; dielectric tube;
120 Carrier gas in
130 Housing for covering high voltage electrode and conduit(s)
140 Second electrode(s); Ground electrode (s)
150 Power supply
160 Distance of first ground electrode from the end of first electrode
165 Distance of the furthest ground electrode from the end of the first electrode
170 Distance of second ground electrode from the end of second electrode
180 Distance below the furthest ground electrode and end of the conduit (outlet)
185 Plasma jet output containing hydrogen peroxide and other reactive species
190 Width of the ground electrode
200 Wound infected with biofilm/cancer site
210 Hydrogel/dressing
220 Trajectory of antimicrobial agents
230 Hydrogen peroxide formed from plasma
240 Antimicrobials loaded into hydrogels
250 Released therapeutic agents

FIG. 1A shows a schematic of the single jet plasma device, of the present invention including a first (high voltage) electrode, 100; a conduit covering the first electrode, 110; an inlet for the flow of carrier gas, 120 in fluid communication with a reservoir including flowmeter and the carrier gas reservoir (not shown). Water vapour molecules could be introduced to the housing, 130 by combining with the carrier gas through the same inlet, 120. The housing 130 contains a longitudinally extending quartz tube, 110, a high voltage electrode (HV electrode), 100 and second (ground) electrode, 140. A plasma generation zone, 160, is provided in the spacing between the HV electrode, 100 and the ground electrode, 140. Plasma and RONS may also be generated between the second (ground) electrode and the end of the conduit towards the output, 180. The positive terminal of the power supply, 150 is connected to the First (HV) electrode, 100 and negative terminal is connected to the second (ground) electrode, 140. Upon application of a sufficient electrical potential across the electrodes, 100, 140, plasma is formed through the ionisation of the carrier gas, and hydrogen peroxide is formed from the dissociation of water molecules present inside the quartz tube, 110. An outlet, 185, is provided for the plasma and the hydrogen peroxide formed. FIG. 1B shows a schematic of the single jet plasma device including a housing 130 in helical configuration. The device includes a first (high voltage) electrode, 100, a second (ground) electrode, 110; an inlet for the flow of carrier gas, 120 in fluid communication with a reservoir including flowmeter and the gas reservoir (not shown). Water vapour molecules would generally be introduced to the housing, 130 with the carrier gas through the same inlet, 120, and the carrier gas is generally combined with water vapour molecules in the reservoir. The housing 130 comprises a helical quartz tube, 110, initially extending along the longitudinal direction of the device, and subsequently extending around the longitudinal axis in a helix. Finally, the housing once again extends along the longitudinal direction of the device towards the outlet, 185. The device also includes a high voltage electrode (HV electrode), 100 and ground electrode, 140. A plasma generation zone, 160, is provided in the spacing between the HV electrode, 100 and the ground electrode, 140. Plasma and RONS may also be generated between the second (ground) electrode and the end of the conduit towards the output, 180. The positive terminal of the power supply, 150 is connected to the first (HV) electrode, 100 and the negative terminal of the power supply is connected to the second ground electrode, 140. Upon application of a sufficient electrical potential across the electrodes, plasma is formed through the ionisation of the carrier gas, and hydrogen peroxide is formed from the dissociation of water molecules present inside the quartz tube, 110. An outlet, 185, is provided for the plasma and the hydrogen peroxide formed.

FIG. 1C shows a schematic of the single jet plasma device, of the present invention including a high voltage electrode, 100; a conduit covering the first electrode, 110; an inlet for the flow of carrier gas, 120 in fluid communication with a reservoir including flowmeter and the gas reservoir (not shown). Water vapour molecules would generally be introduced to the housing, 130 by combining with the carrier gas through1 the same inlet, 120. The housing 130 comprises a longitudinally extending quartz tube, 110. The housing contains a high voltage electrode (HV electrode), 100 and two ground electrodes, 140a, 140b. Plasma generation zones, 160, 170, are provided in the spacing between the HV electrode, 100 and the first ground electrode, 140a, and between the first ground electrode and the second ground electrode, 140b. Plasma and RONS may also be generated between the second ground electrode 140b and the end of the conduit towards the output, 180. The positive terminal of the power supply, 150 is connected to the HV electrode, 100 and negative terminal is connected to 140a, 140b which are connected to ground. Upon application of a sufficient electrical potential across the electrodes, plasma is formed through the ionisation of the carrier gas, and hydrogen peroxide is formed from the dissociation of water molecules present inside the quartz tube, 110. An outlet, 185, is provided for the plasma and the hydrogen peroxide formed.

FIG. 1D shows a schematic of the single jet plasma device in helical configuration, of the present invention including a high voltage electrode, 100; a conduit attached to the top portion of the helical tube, extending perpendicularly towards the top and covering the first electrode, 110; an inlet for the flow of carrier gas, 120 in fluid communication with a reservoir including flowmeter and the gas reservoir (not shown). Water vapour molecules would generally be introduced to the housing, 130 by combining with the carrier gas through the same inlet, 120. The housing 130 comprises a helical quartz tube, 110 extending around the longitudinal axis of the device. There is also provided a high voltage electrode (HV electrode), 100 and first and second ground electrodes, 140a and 140b. Plasma generation zones, 160, 170, are provided in the spacing between the HV electrode, 100 and the first ground electrode, 140a, and between the first ground electrode and the second ground electrode, 140b. Plasma and RONS may also be generated between the second ground electrode 140b and the end of the conduit towards the output, 180. The positive terminal of the power supply, 150 is connected to the HV electrode, 100 and negative terminal is connected to 140a, 140b which are connected to ground. Upon application of a sufficient electrical potential across the electrodes, plasma is formed through the ionisation of the carrier gas, and hydrogen peroxide is formed from the dissociation of water molecules present inside the quartz tube, 110. The bottom end of the helical tube is connected to another quartz tube extending perpendicularly in the vertical direction towards the bottom. An outlet, 185, is provided for the plasma and the hydrogen peroxide formed.

FIG. 1E shows a device of the present invention including a plurality of conduits, 110, with a plurality of high voltage needle electrodes 100 extending in a longitudinal direction along the housing, 130. For communication with the gas reservoir, the device includes a plurality of inlets, 120. Water vapour molecules could be introduced to the housing, 130 by combining with the carrier gas through the same inlet, 120. The housing 130 comprises a plurality of quartz tubes extending in longitudinal direction, 110, a plurality of the high voltage electrodes (HV electrodes), 100 and ground electrodes, 140a, 140b. Plasma generation zones, 160, 170, are provided in the spacing between the HV electrode, 100 and the first ground electrode, 140a, and between the first ground electrode and the second ground electrode, 140b. Plasma and RONS may also be generated between the second ground electrode 140b and the end of the conduit towards the output, 180. The positive terminal of the power supply, 150 is connected to the HV electrodes, 100 and negative terminal is connected to 140a, 140b which are connected to ground. Upon application of a sufficient electrical potential across the electrodes, plasma is formed through the ionisation of the carrier gas, and hydrogen peroxide is formed from the dissociation of water molecules present inside the quartz tube, 110. A plurality of the outlets, 185, are provided in each conduits for the plasma and the hydrogen peroxide formed.

FIG. 1F shows a schematic of the plasma device including a plurality of conduits, 110, some of which are in a helical configuration and some of which extend along the longitudinal axis of the device. There is also provided a plurality of high voltage needle electrodes 100 extending in a longitudinal direction along the housing, 130. The conduits are attached to the top portion of the helical tube, extending perpendicularly towards the top and covering each first electrodes, 110. A plurality of the inlets for the flow of carrier gas, 120 is provided for each conduits through fluid communication with a reservoir including flowmeter and the gas reservoir (not shown). Water vapour molecules could be introduced to the housing, 130 by combining with the carrier gas through the same inlet, 120. The housing 130 contains helical and/or straight conduits extending in longitudinal direction along its axis, 110, a high voltage electrode (HV electrodes), 100 and ground electrodes, 140. Plasma generation zones, 160, 170, are provided in the spacing between the HV electrode, 100 and the first ground electrode, 140a, and between the first ground electrode and the second ground electrode, 140b. Plasma and RONS may also be generated between the second ground electrode 140b and the end of the conduit towards the output, 180. The positive terminal of the power supply, 150 is connected to the HV electrode, 100 and negative terminal is connected to 140a, 140b which are connected to ground. Upon application of a sufficient electrical potential across the electrodes, plasma is formed through the ionisation of the carrier gas, and hydrogen peroxide is formed from the dissociation of water molecules present inside the tubes, 110. Bottom end of each helical tubes are connected to another straight tube extending perpendicularly in the vertical direction towards the bottom. A plurality of the outlets, 185, are provided in each conduits for the plasma and the hydrogen peroxide formed.

FIG. 1G shows the device of FIG. 1E. A plasma composition including reactive oxygen species (including hydrogen peroxide) and reactive nitrogen species are emitted from the outlets, 185. The plasma composition contacts a hydrogel dressing, 210 provided on the surface of a wound infected with a biofilm, 200. The hydrogel dressing, 210 may be loaded with antimicrobial agents, 240 which are activated with the hydrogen peroxide, 230 released from the plasma. Additional therapeutic agents, 250 are released through activation reactions, which along with hydrogen peroxide travel inside the wound and contribute in wound healing through trajectory 220.

In another embodiment of FIG. 1G, the hydrogel dressing may be removed and the wound with biofilm, 200 may be a cancer tumor.

### EXAMPLE 1

### Production of Hydrogen Peroxide by using a Plasma Device with a Single Ground Electrode in a Linear Quartz Tube (For Producing Hydrogen Peroxide in Plasma Treated Water)

The schematic of the plasma device used for this example is shown in FIG. 1A. The fabrication of the plasma jet was done by inserting a stainless-steel needle (outer diameter: 0.90 mm, inner diameter: 0.60 mm, length: 51 mm) inside a quartz tube (inner diameter: 1.5 mm and outer diameter: 3 mm). This needle act as a high voltage electrode/first electrode. The second electrode made up of copper (width: 4 mm, thickness: 1 mm) was positioned at 17 cm below the tip of the high voltage needle electrode. The distance of 17 cm in this plasma jet was specifically determined through a separate experiment conducted to study the effect of inter-electrode spacing (varying from 0.5 cm to 17 cm) on the concentration of hydrogen peroxide. This was tested by constructing plasma jets of different tube lengths (results not shown in this patent) and the plasma jet with higher inter-electrode spacing generated highest concentration of hydrogen peroxide. The first electrode was connected to the positive terminal of the high voltage, high frequency power supply and the second electrode was grounded. The working gas was 99.99% pure argon gas regulated at a constant gas flow rate of 1.2 litres per minute (velocity: 11.32 m/s, Reynold's number: 1340) by using a rotameter. In this configuration, plasma was generated with an applied voltage of 10 kV p-p and frequency of 23.5 kHz.

The argon plasma jet was targeted to a 96-well plate filled with 350 μL of DI water. The distance from the end of the quartz tube to the top of the water surface was fixed to 3 mm. The concentration of hydrogen peroxide formed in the plasma activated water was measured by using commercially available OPD/HRP kit. For this, a calibration curve with known concentrations of H₂O₂ was constructed to determine the concentration of H₂O₂ formed by the plasma jet inside de-ionized water. The measurement kit employed was a mixture of o-phenylenediamine (OPD, CAS number: 95-54-5, Sigma Aldrich Corporation) and horseradish peroxidase (HRP, CAS number: 9003-99-0, Sigma Aldrich Corporation). In the presence of HRP, OPD reacts with H₂O₂ to form 2-3-diaminophenazine which gives fluorescence at 450 nm. For measuring the H₂O₂ concentration, an OPD tablet included in the kit was dissolved in 10 mL of DI water and 20 μL of HRP was added onto the solution. 5 μL of different concentrations of H₂O₂ were added onto different wells of a 96-well plate containing 195 μL of dissolved OPD/HRP and incubated for 15 minutes. The absorbance values measured with the plate reader (λ=450 nm) were proportional to the concentration of H₂O₂ and the obtained line of best fit was used to estimate the H₂O₂ concentration formed in plasma activated water.

The concentration of hydrogen peroxide produced in plasma treated deionized water at different plasma exposure time is shown in FIG. 2A. As the plasma exposure time was increased from 1 minute to 5 minutes, the averaged concentration of hydrogen peroxide also increases from 1.25 mM to 18.90 mM. Increasing the plasma exposure time also increases the interaction of plasma species (mostly electrons, ions, streamers, UVs, etc.) generated inside/outside of the quartz tube with the water molecules present inside/outside of the quartz tube and/or in the well plate. The water molecules are dissociated into hydroxyl radicals which further combine to form hydrogen peroxide.

A photograph of the plasma jet operated with the description as mentioned in this example is shown in FIG. 2B.

### EXAMPLE 2

### Production of Hydrogen Peroxide by using a Plasma Device with a Single Ground Electrode in a Helical Tube (For Producing Hydrogen Peroxide in Plasma Treated water)

A helical quartz tube (inner diameter: 1.5 mm, outer diameter: 3 mm, helical diameter: 15 mm, pitch: 23 mm) as shown in FIG. 1B was constructed to generate plasma and measure the concentration of hydrogen peroxide in plasma treated deionized water. The top and bottom end of the helical tube were connected to quartz tubes which were perpendicular to the axis of the helix and their dimensions were identical to the helical tube. The fabrication of the plasma jet was done by inserting a stainless-steel needle (outer diameter: 0.90 mm, inner diameter: 0.60 mm, length: 30 mm) inside the top portion of the quartz tube. This needle act as a high voltage electrode/first electrode. The second electrode made up of copper (width: 4 mm, thickness: 1 mm) was positioned at 17 cm below the tip of the high voltage needle electrode. The plasma operation conditions and hydrogen peroxide detection methods were identical as described in Example 1. FIG. 3A shows the concentration of hydrogen peroxide as measured from the plasma prepared by treating 350 μL of deionized water in a 96-well plate at 3 mm gap distance below the end of the quartz tube. The concentration of hydrogen peroxide is seen to increase with the increase in the plasma exposure time from 1 minute to 5 minute. At 5 minutes plasma exposure time, the concentration of hydrogen peroxide is ca. 16.5 mM. Increasing the plasma exposure time also increases the interaction of plasma species (mostly electrons, ions, streamers, UVs, etc.) generated inside/outside of the quartz tube with the water molecules present inside/outside of the quartz tube and/or in the well plate. The water molecules are dissociated into hydroxyl radicals which further combine to form hydrogen peroxide. A photograph of the plasma jet operated with the description as mentioned in this example is shown in FIG. 3B.

### EXAMPLE 3

### Production of Hydrogen Peroxide by using a Plasma Device with Two Ground Electrodes in a Linear Quartz Tube (For Activating Hydrogels and Treatment of Wounds in Patients)

The plasma device is intended to generate plasma and hydrogen peroxide for treatment of wounds in patients through activation of hydrogels loaded with therapeutic agents. The schematic of the plasma device used for this example is shown in FIG. 1C. The fabrication of the plasma jet was done by inserting a stainless-steel needle (outer diameter: 0.90 mm, inner diameter: 0.60 mm, length: 51 mm) inside a quartz tube (inner diameter: 1.5 mm and outer diameter: 3 mm). This needle act as a high voltage electrode/first electrode. The first ground electrode made up of copper (width: 4 mm, thickness: 1 mm) was positioned at 5.6 cm below the tip of the high voltage needle electrode. The second ground electrode with identical dimensions as the first one was placed at 5.4 cm below the first ground electrode. These distances were specifically determined to operate the plasma at room temperature. Though the hydrogen peroxide was produced in all regions below the high voltage electrode, the second electrode placed in this configuration helps the plasma plume to reach outside of the quartz tube. The first electrode (stainless steel needle) was connected to the positive terminal of the high voltage, high frequency power supply and both of the second electrodes were grounded. The working gas was 99.99% pure argon gas regulated at a constant gas flow rate of 1.2 litres per minute (velocity: 11.32 m/s, Reynold's number: 1340) by using a rotameter. In this configuration, plasma was generated with an applied voltage of 7 kV p-p and frequency of 23.5 kHz. The plasma generated in this condition is at room temperature.

The argon plasma jet was targeted to a 96-well plate filled with 350 μL of DI water. The distance from the end of the quartz tube to the top of the water surface was fixed to 3 mm. The concentration of hydrogen peroxide formed in the plasma activated water was measured by using commercially available OPD/HRP kit. For this, a calibration curve with known concentrations of H₂O₂ was constructed to determine the concentration of H₂O₂ formed by the plasma jet inside de-ionized water. The measurement kit employed was a mixture of o-phenylenediamine (OPD, CAS number: 95-54-5, SigmaAldrich Corporation) and horseradish peroxidase (HRP, CAS number: 9003-99-0, SigmaAldrich Corporation). In the presence of HRP, OPD reacts with H₂O₂ to form 2-3-diaminophenazine which gives fluorescence at 450 nm. For measuring the H₂O₂ concentration, an OPD tablet included in the kit was dissolved in 10 mL of DI water and 20 μL of HRP was added onto the solution. 5 μL of different concentrations of H₂O₂ were added onto different wells of a 96-well plate containing 195 μL of dissolved OPD/HRP and incubated for 15 minutes. The absorbance values measured with the plate reader (λ=450 nm) were proportional to the concentration of H₂O₂ and the obtained line of best fit was used to estimate the H₂O₂ concentration formed in plasma activated water.

The concentration of hydrogen peroxide produced in plasma treated deionized water at different plasma exposure time is shown in FIG. 4A. As the plasma exposure time is increased from 1 minute to 5 minutes, the averaged concentration of hydrogen peroxide also increases from 0.75 mM to 3.34 mM. Increasing the plasma exposure time also increases the interaction of plasma species (mostly electrons, ions, streamers, UVs, etc.) generated inside/outside of the quartz tube with the water molecules present inside/outside of the quartz tube and/or in the well plate. The water molecules are dissociated into hydroxyl radicals which further combine to form hydrogen peroxide.

A photograph of the plasma jet operated with the description as mentioned in this example is shown in FIG. 4B.

### Example 4

### Production of hydrogen peroxide by using a plasma device with two ground Electrodes in a Helical Quartz Tube (For Activating Hydrogels and Treatment of Wounds in Patients)

A helical quartz tube (inner diameter: 1.5 mm, outer diameter: 3 mm, diameter: 15 mm, pitch: 23 mm) as shown in FIG. 1D was constructed to generate plasma and hydrogen peroxide for treatment of wounds in patients through activation of hydrogels loaded with therapeutic agents. The top and bottom end of the helical tube were connected to quartz tubes which were perpendicular to the axis of the helix and their dimensions were identical to the helical tube. The fabrication of the plasma jet was done by inserting a stainless-steel needle (outer diameter: 0.90 mm, inner diameter: 0.60 mm, length: 30 mm) inside the top portion of the quartz tube. This needle act as a high voltage electrode/first electrode. The first ground electrode made up of copper tape (width: 4 mm) was positioned at 5.6 cm below the tip of the high voltage needle electrode outside of the helical tube. The second ground electrode identical to the first one was placed at 5.4 cm below the first ground electrode. Plasma was generated with an applied voltage of 7 kV p-p and frequency of 23.5 kHz. The plasma generated in this condition is at room temperature. The other parameters for plasma generation and hydrogen peroxide detection were the same as described in Example 3. FIG. 5A shows the concentration of hydrogen peroxide as measured from the plasma treated prepared by treating 350 μL, of deionized water in a 96-well plate at 3 mm gap distance below the end of the quartz tube. The concentration of hydrogen peroxide is seen to increase with the increase in the plasma exposure time from 1 minute to 5 minute. At 5 minutes plasma exposure time, the concentration of hydrogen peroxide is ca. 4.10 mM. Increasing the plasma exposure time also increases the interaction of plasma species (mostly electrons, ions, streamers, UVs, etc.) generated inside/outside of the quartz tube with the water molecules present inside/outside of the quartz tube and/or in the well plate. The water molecules are dissociated into hydroxyl radicals which further combine to form hydrogen peroxide.

A photograph of the plasma jet operated with the description as mentioned in this example is shown in FIG. 5B.

### EXAMPLE 5

### Production of Additional Reactive Species having Biocidal/Virucidal Effects

By controlling the electrode distance, applied voltage, working/surrounding gas, etc., the plasma source can be used for the production of nitrates, nitrites, peroxynitrates, etc. All these reactive species also possess strong biocidal and virucidal effects. The measurement of nitrites was performed by using the Griess reagent in the same conditions as in example 1 for the plasma jet with straight tube. It can be seen in FIG. 6 that the concentration of NO₂ increases from 1 μM to 8 μM as the plasma treatment time is increased from 1 minute to 5 minutes. Short lived reactive species including atomic oxygen and nitric oxide-γ has also been detected using the technique of optical emission spectroscopy (not included in the patent). All these species including hydrogen peroxide and derivatives of oxygen/nitrogen (ozone, nitrous acid, peroxynitrate, etc.) can have a strong bactericidal/virucidal effects. Any person skilled in art can think of other reactive species for this purpose.

### EXAMPLE 6

### Heat Tolerance of the “Tissue” Surrogate against Plasma Exposure

Treating thermally sensitive materials without rastering over the surface is an important challenge for successful application of plasma jets in medicine. In this example, the plasma jet as described in example 3 was used to treat an agarose gel which acts as a surrogate for real tissues. 0.5% agarose was heated in DI water and transferred to a petri dish before it solidified to form a gel. Firstly, the conventional helium plasma jet at 4 mm distance below the nozzle of the glass tube was targeted to the agarose gel (FIG. 7(A)). After two minutes of plasma exposure time, the centre of the gel in contact with the plasma jet was observed to be burnt. Next, the argon plasma jet (FIG. 7(B)) as described in example 3 was used to treat the agarose gel. The gel appeared to cope well and no burning was observed until continuous two minutes plasma exposure time at the same point. This suggests that the plasma jet as described in this patent is well suited for treating thermally sensitive materials.

### EXAMPLE 7

### Dimensions of the Plasma Device with a Single Ground Electrode

We investigated the relationship between the ratio of the inner diameter of dielectric tubes (Di) and the spacing between first and second electrodes (d). Dielectric tubes with different inner diameters were constructed. The inner diameters of the tubes ranged from 0.5 to 12 mm (Di). We experimented with different inter-electrode spacings (d) ranging from 20 mm to 50 cm. The experiments were conducted with a device including a single ground electrode, and two ground electrodes. A preferred ratio of Di:d of less than 0.6 was identified. The results are summarised in FIG. 8.

### EXAMPLE 8

### Minimum Volume of the Plasma Generation Zone

We assessed the minimum volume of the plasma generation zone for devices of different inner diameters. The distance between the first electrode (HV) and the second ground electrode (d) or the distance between the first electrode (HV) and the furthest ground electrode (d₂) were varied from 4 to 20 cm. The inner diameters of the tubes were varied from 0.5 mm to 12 mm. We created devices having 1, 7 and 9 conduits (or jets) formed from these dielectric tubes. The results are summarised in the table below.

| | Dᵢ | d or d₂ | 1-jet | 7-jet | 9-jet |
|---|---|---|---|---|---|
| Range | [cm] | [cm] | [cm³] | [cm3] | [cm3] |
| Smallest | 0.05 | 4 | 0.007854 | 0.0549779 | 0.0706858 |
| | 0.05 | 10 | 0.019635 | 0.1374447 | 0.1767146 |
| | 0.05 | 15 | 0.0294524 | 0.206167 | 0.2650719 |
| | 0.05 | 20 | 0.0392699 | 0.2748894 | 0.3534292 |
| Typical | 0.15 | 4 | 0.0706858 | 0.4948008 | 0.6361725 |
| | 0.15 | 10 | 0.1767146 | 1.2370021 | 1.5904313 |
| | 0.15 | 15 | 0.2650719 | 1.8555032 | 2.3856469 |
| | 0.15 | 20 | 0.3534292 | 2.4740042 | 3.1808626 |
| | 0.5 | 4 | 0.7853982 | 5.4977871 | 7.0685835 |
| | 0.5 | 10 | 1.9634954 | 13.744468 | 17.671459 |
| | 0.5 | 15 | 2.9452431 | 20.616702 | 26.507188 |
| | 0.5 | 20 | 3.9269908 | 27.488936 | 35.342917 |
| Largest | 1.2 | 4 | 4.5238934 | 31.667254 | 40.715041 |
| | 1.2 | 10 | 11.309734 | 79.168135 | 101.7876 |
| | 1.2 | 15 | 16.9646 | 118.7522 | 152.6814 |
| | 1.2 | 20 | 22.619467 | 158.33627 | 203.5752 |

### EXAMPLE 9

### Comparison of Plasma Jets with One (E₁), Two (E₁+E₂+E₃) and Three (E₁+E₂+E₃) Ground Electrodes with Linear and Helical Designs

The schematic of the plasma device with more than one ground electrode (in linear configuration) is shown in FIG. 9. This schematic was applied to develop plasma devices up to three ground electrodes in linear and helical configurations. These are shown in FIG. 10A to 10F. For both linear and helical configurations of the tube, a stainless steel needle of length 22 mm was inserted inside the quartz tubes through the top end. This needle acted as a high voltage electrode/first electrode. The first ground electrode was formed from copper (width: 5 mm, thickness: 1 mm), positioned 5 cm below the tip of the high voltage needle electrode (FIG. 10A and 10D). A second ground electrode with identical dimensions to the first ground electrode described above was placed at 5.1 cm below the first ground electrode (FIG. 10B and 10E). The third ground electrode, with identical dimensions to the first ground electrode described above, was positioned at 3.5 cm below the second ground electrode (FIG. 10C and 10F). The total length of both the linear and helical tubes below the high voltage electrode/ first electrode was 23 cm. The working gas was 99.99% pure argon gas regulated at a constant gas flow rate of 1.0 litres per minute by using a rotameter. In this configuration, plasma was generated with an applied voltage of 7 kV p-p and frequency of 23.5 kHz. The photograph provided in FIG. 10A and 10D show that with one ground electrode, most of the plasma is inside the quartz tube. The addition of a second ground electrode increases the length of the plasma outside of the quartz tube FIG. 10B and 10E. With the addition of the third ground electrode, the length of the plasma plume is slightly increased (see FIG. 10C and 10F).

The concentration of H₂O₂ and temperature of all the plasma jets as shown in FIGS. 11A to 11F were also compared. The plasma jets were targeted to a 96-well plate filled with 350 μL of DI water. The distance from the end of the quartz tube to the top of the water surface was fixed to 3 mm. The concentration of hydrogen peroxide formed in the plasma activated water was measured by using commercially available o-phenylenediamine (OPD)/horseradish peroxidase (HRP) kit. For this, a calibration curve with known concentrations of H₂O₂ was constructed to determine the concentration of H₂O₂ formed by the plasma jet inside de-ionized water. The measurement kit employed was a mixture of o-phenylenediamine (OPD, CAS number: 95-54-5, SigmaAldrich Corporation) and horseradish peroxidase (HRP, CAS number: 9003-99-0, SigmaAldrich Corporation). In the presence of HRP, OPD reacts with H₂O₂ to form 2-3-diaminophenazine which gives fluorescence at 450 nm. For measuring the H₂O₂ concentration, an OPD tablet included in the kit was dissolved in 10 mL of DI water and 20 μL of HRP was added onto the solution. 20 μL of different concentrations of H₂O₂ were added onto different wells of a 96-well plate containing 180 μL of dissolved OPD/HRP and incubated for 15 minutes. The absorbance values measured with the plate reader (μ=450 nm) were proportional to the concentration of H₂O₂ and the obtained line of best fit was used to estimate the H₂O₂ concentration formed in plasma activated water.

The concentration of hydrogen peroxide produced in plasma treated deionized water at plasma exposure time of 2 minutes is shown in FIG. 11A for both linear and helical configurations. For linear plasma jet, the concentration of H₂O₂ with one ground electrode (E₁) is ca. 167 μM. With the addition of a second ground electrode (E₁+E₂), the concentration is increased to ca. 423 μM. This further increased to ca. 585 μM with the addition of a third ground electrode (E₁+E₂+E₃).

A similar trend is observed with the helical configuration but the concentrations were slightly lower than the linear configuration.

The temperatures of the plasma jets were measured with a mercury at 5 mm position below the end of the quartz tube. The bulb of the thermometer was wrapped by a tape and it was treated by the plasma jet continuously for up to one minute. The readings at the end of plasma treatment were noted. The results are shown in FIG. 11B. The temperature of the target point increases slightly with the addition of second and third ground electrodes for both linear and helical configurations. With three ground electrodes, the temperatures of linear and helical jets are found to be 42.5 and 34 degree Celsius. The slightly lower temperature with the helical jet is attributed to the configuration of the plasma design.

### EXAMPLE 10

### Dimensions of the Plasma Device with More than One Ground Electrode

The schematic of the plasma device with more than one ground electrode (in linear configuration) is represented in FIG. 9. We used this schematic to identify the dimensions of the devices that could be operated by including more than one ground electrode. The inner diameters of the tubes ranged from 0.5 to 12 mm (Di). The dimensions of the devices could be summarised in terms of two parameters: (i) ratio of inner diameter of the tube (D₁) to the distance between the first electrode (high voltage, HV) and the closest ground electrode (d₁) (=Di/d₁), (ii) ratio of inner diameter of the tube (Dᵢ) to the distance between the first electrode (HV) and the furthest ground electrode (d₂), (=Di/ d₂). The results are summarised in FIG. 12A and 12B.

### EXAMPLE 11

### A Multi-Jet Plasma Device in Conical Configuration and its Temperature Measurement

FIG. 13A shows the schematic of the conical device of the present invention including a plurality of conduits, 110, with a plurality of high voltage needle electrodes 100 extending in a longitudinal direction along the housing, 130. For communication with the gas reservoir, the device includes a plurality of inlets, 120. Water vapour molecules could be introduced to the housing, 130 by combining with the carrier gas through the same inlet, 120. The housing 130 comprises a plurality of quartz tubes extending in longitudinal direction, 110, a plurality of the high voltage electrodes (HV electrodes), 100 and ground electrodes, 140a, 140b. Plasma generation zones, 160, 170, are provided in the spacing between the HV electrode, 100 and the first ground electrode, 140a, and between the first ground electrode and the second ground electrode, 140b. Plasma and RONS may also be generated between the second ground electrode 140b and the end of the conduit towards the output, 180. The positive terminal of the power supply, 150 is connected to the HV electrodes, 100 and negative terminal is connected to 140a, 140b which are connected to ground. Upon application of a sufficient electrical potential across the electrodes, plasma is formed through the ionisation of the carrier gas, and hydrogen peroxide is formed from the dissociation of water molecules present inside the quartz tube, 110. A plurality of the outlets, 185, are provided in each conduits for the plasma and the hydrogen peroxide formed.

The schematic in FIG. 13A was utilised to fabricate a conical configuration of the plasma device. This is shown in FIG. 13B. It consists of seven individual quartz tubes (inner diameter: 1.5 mm, outer diameter: 3 mm) inserted inside seven holes of a conical housing (inner diameter: 3.20 mm). A high voltage needle electrode (stainless steel) was inserted inside each quartz tubes and sealed inside the housing using torr-seal. Ground electrodes made up of copper (thickness: 1 mm) were wrapped outside each of the seven tubes at positions of 30 mm and 60 mm below the tip of the high voltage electrode. The distance of the each nozzles of the quartz tubes from the end of second ground electrode was 30 mm. FIG. 14B (left) shows the photographs of the individual plasma jets operated with argon gas and FIG. 14B (right) shows the photograph of the device with all 7-jets operated together. The operating parameters for the plasma device were: applied voltage: ˜8 kV p-p, frequency: 23.5 kHz, Ar flow rate: 5 standard litres per minute. The temperature of the plasma plume was measured using an infrared thermometer (bottom photograph on the right) and it was found to be less than 30 degree Celsius. This was true for all seven plasma jets operated individually or together. This confirms that the conical design of the multi-jet plasma device could be suitable for the treatment of thermally sensitive materials including hydrogel dressings.

The conical design of the multi-jet would reduce the interaction of the individual jets with each other and reduce electrode heating thereby operating the plasma jet at room temperature. The temperature of the jet would further be reduced by operating the jet in dimming mode.

Various modifications and variations of the described aspects of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the relevant fields are intended to be within the scope of the following Claims.

1. A plasma device for the generation of reactive oxygen, nitrogen species (RONS), the device comprising:
a reservoir containing a carrier gas;
a housing in fluid communication with the reservoir, wherein the reservoir includes air or water, or the housing includes an air or water inlet, said housing comprising at least one conduit formed from a dielectric material;
first and second electrodes spaced along the at least one conduit wherein a plasma generation zone is provided in the spacing between the first and second electrodes;
a power supply suitable to apply an electrical potential between the first and second electrodes sufficient to form a plasma through the ionisation of the carrier gas, and to form reactive oxygen nitrogen species from the water molecules; and
at least one outlet for the plasma and the reactive oxygen nitrogen species formed.

2. The device of claim 1, wherein the spacing between the first and second electrodes is more than 2 to 100 cm, there is a spacing between the one of the first and second electrodes closest to the outlet and the outlet of at least 0.5 cm, and wherein the or each conduit has an associated inner diameter or minimum inner width, and the ratio of the inner diameter or inner width of the or each conduit (Di) to the spacing between the first and second electrodes (d) is 0.0005 to 0.6, suitably wherein the spacing between the first and second electrodes is 15 to 20 cm and the ratio Di/d is 0.002 to 0.08.

3. The device of claim 2, wherein the inter-electrode spacing is more than 2 cm and up to 20 cm and the ratio Di to d is 0.0025 to 0.6 or wherein the inter-electrode spacing is 10 to 30 cm and the ratio Di to d is 0.001 to 0.12.

4. (canceled)

5. The device of claim 2, wherein the inter-electrode spacing is 30 to 50 cm and the ratio Di to d is 0.001 to 0.04, or wherein the inter-electrode spacing is 50 to 100 cm and the ratio Di to d is 0.00025 to 0.03, generally 0.0005 to 0.024.

6. (canceled)

7. The device of claim 1, wherein the housing tapers along its length towards the outlet resulting in a substantially conical configuration.

8. The device of claim 1, wherein the first electrode is connected to the positive terminal of the power supply and the second electrode is connected to the negative terminal of the power supply, and wherein the first electrode is provided within the conduit, and the second electrode is provided externally to the conduit, generally abutting an outer wall of the conduit.

9. (canceled)

10. The device of claim 1, wherein:
the housing has a length, a first end towards the reservoir and a second end towards the outlet;
the ratio of the inner diameter or inner width of the, or each conduit (Di) to the spacing between the first and second electrodes (d) is 0.0005 to 0.3; and
the length of the housing between the first electrode and the second end is 11 to 25 cm, where there is a spacing of at least 0.5 cm between the second electrode and the second end.

11. The device of claim 1 including more than one electrode connected to the negative terminal of the power supply or the ground, suitably two or three electrodes connected to the negative terminal of the power supply or the ground, or wherein each of the more than one electrode connected to the negative terminal of the power supply is provided externally to the conduit, generally abutting an outer wall of the conduit.

12. (canceled)

13. The device of claim 11, wherein the first electrode is connected to the positive terminal of the power supply, the spacing between the first electrode and the ground electrode furthest therefrom is 50 to 100 cm and the ratio of the inner diameter or inner width of the or each conduit (Di) to the spacing between the first (high voltage) electrode and the ground electrode furthest from the first electrode is 0.0005 to 0.024.

14. The device of claim 10, wherein:
the housing has a length, a first end towards the reservoir and a second end towards the outlet,
the at least one conduit has an associated inner diameter or inner width of 0.5 to 5 mm,
a first (high voltage) electrode is provided within the at least one conduit and more than one ground electrode is spaced along an outer surface of the at least one conduit, wherein a plasma generation zone is provided in the spacing along the length of the conduit between the first (high voltage) electrode and the ground electrode closest to the first (high voltage) electrode, wherein the spacing between the first (high voltage) electrode and the ground electrode closest to it is 2 to 7 cm and the spacing between the first (high voltage) electrode and the ground electrode furthest from it is 4 to 100 cm;
the power supply is suitable to apply an electrical potential between the first (high voltage) electrode and the ground electrode closest to the first (high voltage) electrode sufficient to form a plasma through the ionisation of the carrier gas, and to form RONS such as hydrogen peroxide through the ionisation of the water molecules and optionally through ionisation of air; and
the length of the housing between the first (high voltage) electrode and the end of the housing towards the at least one outlet is 11 to 25 cm, and there is a spacing of 0.5 to 2 cm between the ground electrode closest to the at least one outlet and the end of the housing towards the at least one outlet.

15. The device as claimed in claim 1 wherein the section of the conduit or conduits between the first and second electrodes forms a helix around an axis of the plasma device extending in a longitudinal direction or undulates in a wave form along an axis of the plasma device extending in a longitudinal direction.

16. The device of claim 1, wherein the housing includes more than one conduit, and the first and second electrodes are spaced along the more than one conduit wherein the plasma generation zone is provided within the more than one conduit in the spacing between the first and second electrodes, or wherein the housing comprises one to thirteen of the conduits.

17. The device of claim 16, wherein some or all of the conduits extend in a longitudinal direction along the housing, along or parallel to the longitudinal axis of the housing, and/or wherein some or all of the conduits form a helix around an axis of the housing extending in a longitudinal direction.

18. (canceled)

19. The device of claim 1 including more than one outlet for the plasma and the reactive oxygen nitrogen species.

20. A method of forming a plasma including reactive oxygen nitrogen species, the method comprising:
providing the device of claim 1;
providing a flow of the carrier gas comprising water molecules through the housing; and
applying an electrical potential between the first and second electrodes to ionise the carrier gas to form a plasma and reactive oxygen nitrogen species, and
wherein: the electrical potential is applied at a voltage of 0.5 to 30 kV(rms) and at a frequency of 1 kHz to 1 MHz.

21. (canceled)

22. The method of claim 20 including providing a surrounding gas between the electrode closest to the at least one outlet and the end of the housing towards the at least one outlet wherein the surrounding gas is selected from the group consisting of argon, helium, nitrogen, oxygen and mixtures thereof.

23. A plasma treatment method for a patient in need thereof, the method comprising:
providing a hydrogel on an anatomical region of interest of the patient;
generating a plasma comprising reactive oxygen nitrogen species using the device of claim 1; and
contacting a surface of the hydrogel with the plasma comprising reactive oxygen nitrogen species; wherein contact of the hydrogel with the plasma activates the hydrogel dressing.

24. The method of claim 23, wherein the hydrogel includes therapeutic agents and activation of the hydrogel causes release of the therapeutic agents, and the anatomical region of interest is one or more of a wound, an infected area (for instance by micro-organisms or parasites) and a burn.

25. (canceled)

26. A method of deactivation of micro-organisms on a surface, the method comprising:
generating a plasma comprising reactive oxygen nitrogen species using the device of claim 1;
contacting the surface with the plasma comprising hydrogen peroxide,
wherein the plasma has a temperature of 30 to 40° C., and is emitted from the device over an area of 0.5 cm² to 10 cm², generally 3 to 7 cm² wherein the concentration of RONS in the plasma is from 1 to 1000 mM.

27. (canceled)

28. A system including the device claim 1 and a hydrogel dressing comprising therapeutic agents wherein the hydrogel dressing is activatable upon contact with a plasma comprising reactive oxygen nitrogen species to release the therapeutic agents.

29-30. (canceled)
